(19) United States
(12) Patent Application Publication
(10) Pub. No.: US 2025/0320547 A1
(43) Pub. Date: Oct. 16, 2025

(54) NUCLEIC ACID ABSOLUTE QUANTIFICATION SYSTEM AND METHOD

(71) Applicant: SHENZHEN UNIVERSITY, Shenzhen (CN)

(72) Inventors: Wenwen CHEN, Shenzhen (CN); Xi LU, Shenzhen (CN); Jidong WANG, Shenzhen (CN)

(21) Appl. No.: 19/201,099

(22) Filed: May 7, 2025

Related U.S. Application Data
(63) Continuation of application No. PCT/CN2022/134280, filed on Nov. 25, 2022.

(30) Foreign Application Priority Data
Nov. 7, 2022 (CN) 202211387006.X
Apr. 21, 2025 (CN) 202510501776.X

(51) Int. Cl. C12Q 1/6851; C12Q 1/48; C12Q 1/686; C12Q 1/689

(52) U.S. Cl. CPC C12Q 1/6851; C12Q 1/48; C12Q 1/686; C12Q 1/689; C12Y 207/07

(57) ABSTRACT

A nucleic acid absolute quantification system, including a polyethylene glycol acrylate compound or a polyethylene glycol maleimide compound, a polyethylene glycol-thiol compound, a primer for the target nucleic acid molecule; a nucleic acid amplification reagent and a fluorescent agent. A mass ratio of the polyethylene glycol acrylate compound or the polyethylene glycol maleimide compound to the polyethylene glycol-thiol compound is 1-30:10-1. A nucleic acid absolute quantification kit and method are also provided.

### CROSS-REFERENCE TO RELATED APPLICATIONS

This application is a continuation of International Patent Application No. PCT/CN2022/134280, filed on Nov. 25, 2022, which claims the benefit of priority from Chinese Patent Application No. 202211387006.X, filed on Nov. 7, 2022; and Chinese Patent Application No. 202510501776.X, filed on Apr. 21, 2025. The content of the aforementioned application, including any intervening amendments thereto, is incorporated herein by reference in its entirety.

### REFERENCE TO AN ELECTRONIC SEQUENCE LISTING

The contents of the electronic sequence listing (Name: SequenceListing.xml; Size: 16,924 bytes; and Date of Creation: May 4, 2025) is herein incorporated by reference in its entirety.

### TECHNICAL FIELD

This application relates to molecular biology technology, and more specifically to a nucleic acid absolute quantification system and method.

### BACKGROUND

Nucleic acids, including DNA and RNA, are capable of encoding genetic information, and thus serve as storage media in the intergenerational transmission of genetic materials. Nucleic acid amplification and quantification are crucial in the molecular biology researches, which have been extensively applied to molecular sequencing, gene expression analysis, gene mutation research, early molecular diagnosis of diseases, single nucleotide polymorphism (SNP) studies, and drug screening.

Currently, the most widely used nucleic acid quantification technology is real-time fluorescent quantitative polymerase chain reaction (qPCR). This method monitors template amplification in real time during detection based on the signal from added fluorescent molecular probes, with results output as linear amplification signals. The initial template copy number of an unknown sample is calculated based on the amplification curve of a known standard sample. Therefore, qPCR is a relative nucleic acid quantification method, and its sensitivity and accuracy are limited. In recent years, digital polymerase chain reaction (dPCR) technology has developed rapidly. This technique isolates individual nucleic acid molecules in separate compartments for PCR reactions to identify the presence of target molecules. Currently, dPCR technology mainly includes microfluidic chip array reaction chambers (or digital droplet analysis technology) and emulsion micro-droplet digital analysis technology. The dPCR technology based on microfluidic device and chip has a limited scalability and a low detection throughput. The emulsion micro-droplet digital analysis technology serves as a high-throughput dPCR technique by means of the sealing of magnetic beads with an emulsion. However, this technology still has several drawbacks, such as failure to detect target templates when the template and magnetic beads are not partitioned into the same droplet, the impact of polymerase inhibitors in DNA extracts on amplification efficiency, and the high complexity of the operation procedure and thermal cycling amplification.

In summary, it is urgently needed for those skilled in the art to develop a nucleic acid absolute quantification method with high throughput, excellent technical stability, and simple operation.

### SUMMARY

Objectives of the present disclosure are to provide a system, kit and method for nucleic acid absolute quantification. The novel nucleic acid absolute quantification system designed herein enables rapid, accurate, simple and cost-effective absolute quantification of nucleic acids without plotting a standard curve.

Technical solutions of the present disclosure are described below.

In a first aspect, the present disclosure provides a nucleic acid absolute quantification system, which is selected from the group consisting of a first system, a second system, a third system, a fourth system, a fifth system, a sixth system and a combination thereof.

The first system comprises a polyethylene glycol acrylate compound containing two or more acrylate groups or a polyethylene glycol maleimide compound containing two or more maleimide groups, a polyethylene glycol-thiol compound containing two or more thiol groups, a primer for a target nucleic acid molecule, a nucleic acid amplification reagent, and a first fluorescent reagent;
wherein a mass ratio of the polyethylene glycol acrylate compound or the polyethylene glycol maleimide compound to the polyethylene glycol-thiol compound is 1-30:10-1;
the polyethylene glycol acrylate compound is selected from the group consisting of a polyethylene glycol diacrylate (AC-PEG-AC), a three-arm polyethylene glycol acrylate (3-arm-PEG-AC), a four-arm polyethylene glycol acrylate (4-arm-PEG-AC), and an eight-arm polyethylene glycol acrylate (8-arm-PEG-AC);
the polyethylene glycol maleimide compound is selected from the group consisting of bismaleimide polyethylene glycol (MAL-PEG-MAL), three-armed polyethylene glycol maleimide (3-arm-PEG-MAL), four-armed polyethylene glycol maleimide (4-arm-PEG-MAL), six-armed polyethylene glycol maleimide (6-arm-PEG-MAL), eight-armed polyethylene glycol maleimide (8-arm-PEG-MAL); and
the polyethylene glycol-thiol compound is a dithiol polyethylene glycol (SH-PEG-SH) or a four-arm polyethylene glycol thiol (4-arm-PEG-SH).

The formation of the PEG hydrogel requires two monomeric molecules, wherein a PEG molecule of the first monomer is required to contain two or more double bonds (including but not limited to acrylate or maleimide groups), and a PEG molecule of the second monomer is required to contain two or more thiol groups. These monomers undergo Michael addition polymerization to form the hydrogel. Consequently, any other molecules satisfying the above requirements can participate in gel formation to construct the PEG-based nucleic acid absolute quantification system, and other molecules are omitted here.

A chemical formula of the polyethylene glycol acrylate compound is

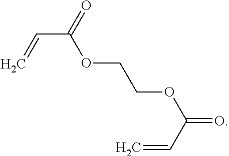

A chemical formula of the 3-arm-PEG-AC is

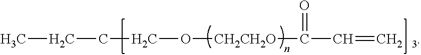

A chemical formula of the 4-arm-PEG-AC is

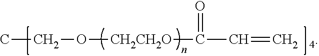

A chemical formula of the 8-arm-PEG-AC is

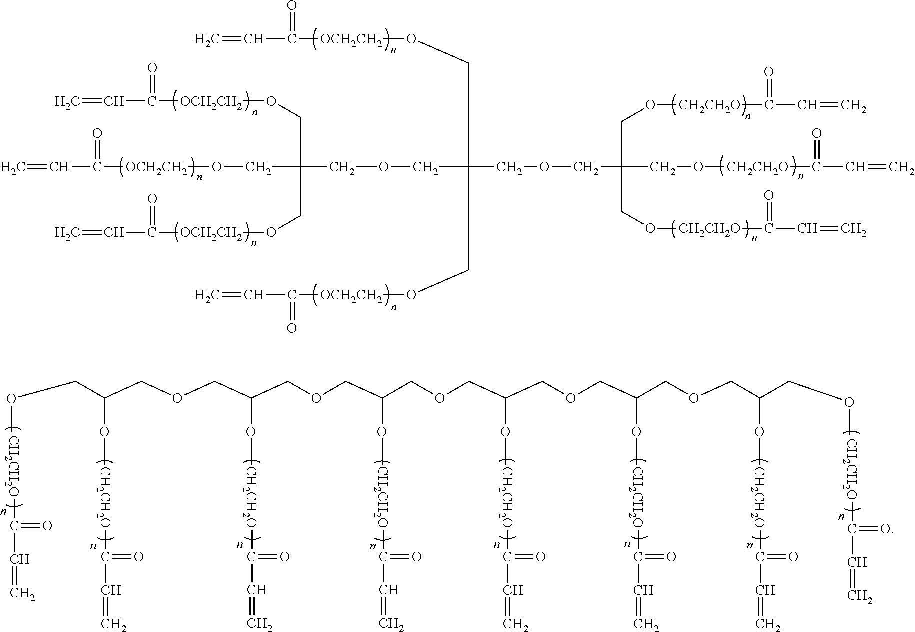

A chemical formula of the polyethylene glycol maleimide compound is

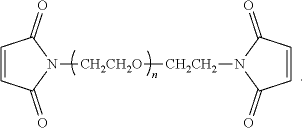

A chemical formula of the 3-arm-PEG-MAL is

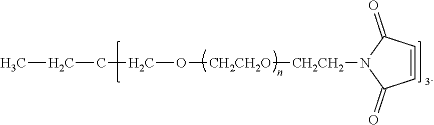

A chemical formula of the 4-arm-PEG-MAL is

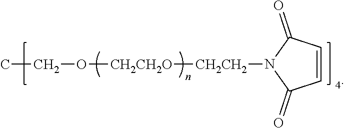

A chemical formula of the 6-arm-PEG-MAL is

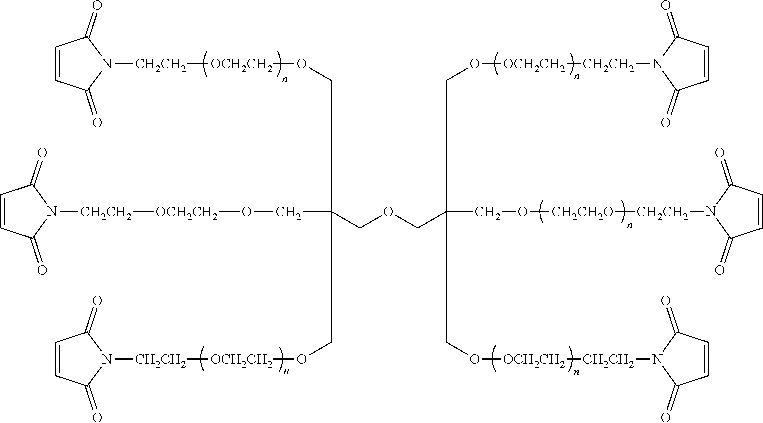

A chemical formula of the 8-arm-PEG-MAL is

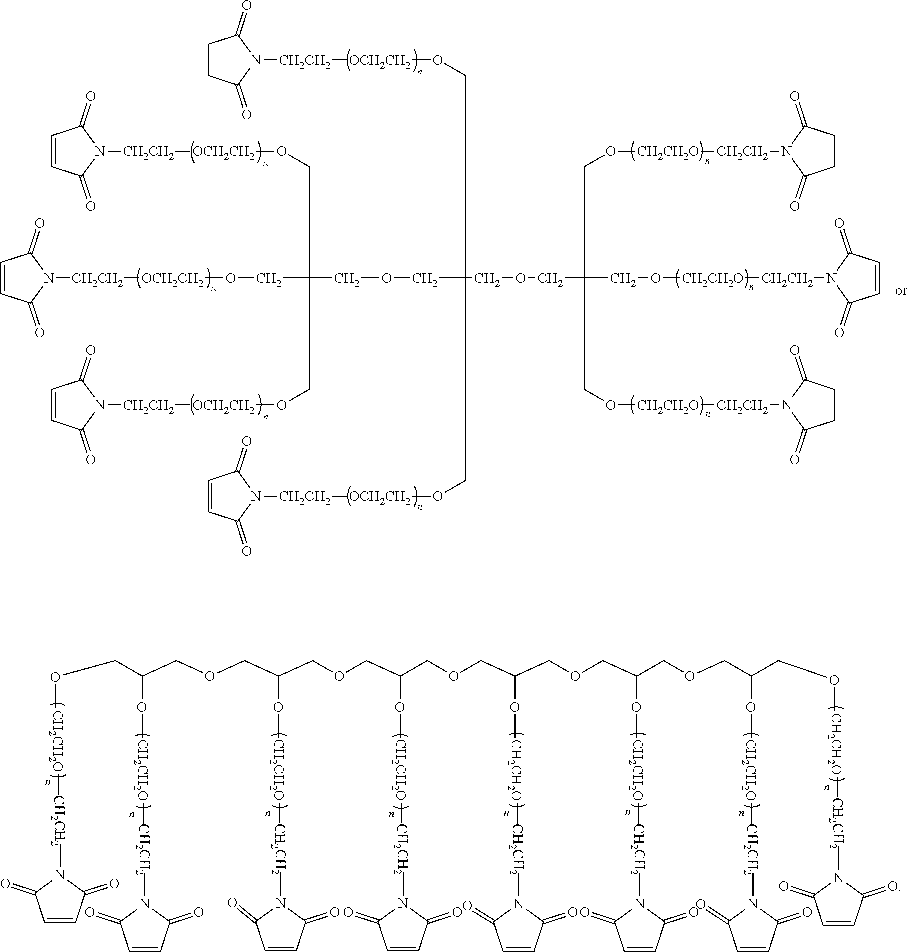

A chemical formula of the polyethylene glycol-thiol compound is

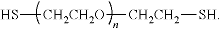

A chemical formula of the 4-arm-PEG-SH is

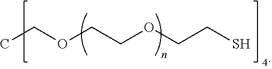

The second system comprises a N-isopropylacrylamide (NIPAM), potassium persulfate (KPS) as initiator, N,N-methylenebisacrylamide (MBAA) as crosslinker, the primer, the nucleic acid amplification reagent, and a second fluorescent reagent; wherein a mass ratio of the NIPAM to the KPS, and to the MBAA is 80-98:0.5-5:0.1-1.

The third system comprises hydroxyethyl methacrylate (HEMA), ethylene glycol dimethacrylate (EGDMA) as crosslinker, ammonium persulfate (APS) as initiator, tetramethylethylenediamine (TMEDA) as co-initiator, the primer, the nucleic acid amplification reagent, and a third fluorescent reagent; wherein a mass ratio of the HEMA to the EGDMA, to the APS, and to the TMEDA is 70-95:0.5-5:0.1-1:0.1-1.

The fourth system comprises acrylamide (AM), polyethylene glycol diacrylate (PEGDA) as crosslinker, 2-hydroxy-2-methylpropiophenone (HMPP) as initiator, the primer, the nucleic acid amplification reagent, and a fourth fluorescent reagent; wherein a mass ratio of the AM to the PEGDA, and to the HMPP is 70-90:5-20:0.1-1.

The fifth system comprises acrylamide (AM), N,N′-methylenebisacrylamide (BIS), ammonium persulfate (APS) as initiator, tetramethylethylenediamine (TMEDA) as crosslinker, the primer, the nucleic acid amplification reagent, and a fifth fluorescent reagent; wherein a mass ratio of the AM to the BIS, to the APS, and to the TMEDA is 152-228:8-12:0.1-2:0.1-2.

The sixth system comprises 2-Acrylamido-2-methylpropane sulfonic acid (AMPS), acrylamide (AM), N,N′-methylenebisacrylamide (BIS) as crosslinker, 2-hydroxy-2-methylpropiophenone (HMPP) as initiator, the primer, the nucleic acid amplification reagent, and a sixth fluorescent reagent; wherein a mass ratio of the AMPS to the AM, to the BIS, and to the HMPP is 1:2-10:1-3:2-4.

The polyethylene glycol acrylate compound and the dithiol polyethylene glycol spontaneously polymerize to form a PEG hydrogel polymer.

The N-isopropylacrylamide (NIPAM), in the presence of the initiator of potassium persulfate (KPS) and the crosslinker of N,N-methylenebisacrylamide (MBAA), self-polymerizes into a PNIPAM hydrogel polymer.

The hydroxyethyl methacrylate (HEMA), with the crosslinker of ethylene glycol dimethacrylate (EGDMA), the initiator of ammonium persulfate (APS), and the co-initiator of tetramethylethylenediamine (TMEDA), spontaneously forms PHEMA hydrogel polymers.

Acrylamide (AM), in the presence of the crosslinker of polyethylene glycol diacrylate (PEGDA) and the initiator of 2-hydroxy-2-methylpropiophenone (HMPP), spontaneously forms PAM hydrogel polymers.

Acrylamide (AM) and N,N′-methylenebisacrylamide (BIS), in the presence of the crosslinker tetramethylethylenediamine (TMEDA) and the initiator of ammonium persulfate (APS), self-assemble into PAM hydrogel polymers.

2-Acrylamido-2-methylpropane sulfonic acid (AMPS) and acrylamide (AM), in the presence of crosslinker N,N′-methylenebisacrylamide (BIS) and initiator 2-hydroxy-2-methylpropiophenone (HMPP), polymerize to form P(AMPS-AM) hydrogel polymers.

Under room temperature conditions, these hydrogel polymers achieve polymerization within minutes.

In some embodiments, the polyethylene glycol acrylate compound is 4-arm-PEG-AC, and the polyethylene glycol-thiol compound is SH-PEG-SH.

The present application designs a nucleic acid absolute quantification system utilizing a PEG hydrogel system formed by two monomers (i.e., 4Arm-PEG-AC and SH-PEG-SH). This system spontaneously polymerizes at room temperature to form a hydrogel without initiators, thereby avoiding interference with the nucleic acid amplification reaction. By controlling the mass ratio of the two monomers, the system further enhances the speed and efficiency of nucleic acid amplification and improves fluorescence diffusion, enabling rapid and accurate quantitative detection.

Additionally, the present application includes alternative hydrogel systems as media for nucleic acid amplification reactions, including but not limited to: a PNIPAM hydrogel system prepared from N-isopropylacrylamide (NIPAM), potassium persulfate (KPS), and N,N-methylenebisacrylamide (MBAA); a PHEMA hydrogel system prepared from hydroxyethyl methacrylate (HEMA), ethylene glycol dimethacrylate (EGDMA), ammonium persulfate (APS), and tetramethylethylenediamine (TMEDA); a PAM hydrogel system prepared from acrylamide (AM), polyethylene glycol diacrylate (PEGDA), and 2-hydroxy-2-methylpropiophenone (HMPP); a PAM hydrogel system prepared from acrylamide (AM), N,N′-methylenebisacrylamide (BIS), tetramethylethylenediamine (TMEDA), and ammonium persulfate (APS); and a P(AMPS-AM) hydrogel system prepared from 2-acrylamido-2-methylpropane sulfonic acid (AMPS), acrylamide (AM), N,N′-methylenebisacrylamide (BIS), and 2-hydroxy-2-methylpropiophenone (HMPP).

By controlling the mass ratios of the components of the hydrogel, the present application achieves a porous hydrogel polymer structure. This structure retains target analytes (e.g., cells, nucleic acids, microorganisms, or blood) within its micropores, while allowing primers and reagents to diffuse through the nanoscale pores.

The microporous hydrogel network restricts the movement of inhibitors and interferents, creating a purer reaction environment compared to liquid-phase systems, and mitigating their adverse effects on the amplification reaction. Within the specified mass ratio ranges, the system delivers optimal detection performance.

In some embodiments, the nucleic acid absolute quantification system comprises: a polyethylene glycol acrylate compound containing two or more acrylate groups or a polyethylene glycol maleimide compound containing two or more maleimide groups, a polyethylene glycol-thiol compound containing two or more thiol groups, a primer for the target nucleic acid molecule, a nucleic acid amplification reagent and a fluorescent reagent, wherein the mass ratio of the polyethylene glycol acrylate compound or the polyethylene glycol maleimide compound to the polyethylene glycol-thiol compound is 1-30:10-1.

In some embodiments, a weight-average molecular weight of the polyethylene glycol acrylate compound or the polyethylene glycol maleimide compound is 5,000-40,000; and/or a weight-average molecular weight of the polyethylene glycol-thiol compound is 1,000-20,000.

In some embodiments, the nucleic acid absolute quantitation system comprises a 4-arm polyethylene glycol acrylate (4-arm PEG-acrylate), a dithiol polyethylene glycol (SH-PEG-SH), a primer for the target nucleic acid molecule and a nucleic acid amplification reagent, wherein the mass ratio of 4-Arm PEG-acrylate to SH-PEG-SH is 1-10:10-1, including but not limited to: 1:1, 1.1:1, 1.2:1, 1.3:1, 1.4:1, 1.5:1, 1.7:1, 1.8:1, 2:1, 3:1, 4:1, 5:1, 6:1, 7:1, 7:3, 8:1, 9:1, 10:1, 1:10, 2:3, 3:4, 3:7, 4:5, 5:6, 6:7, 7:8, 8:9, 9:10, and 16:11.

More preferably, the mass ratio of the 4-arm PEG-Acrylate to the SH-PEG-SH is 1:10, 3:7, 1:1, 7:3, 10:1, or 16:11.

The 4-Arm PEG-acrylate has a multi-arm structure formed by four acrylate functional groups attached to a polyethylene glycol (PEG) core.

The dithiol polyethylene glycol (SH-PEG-SH) is a dithiol compound consisting of two terminal thiol groups connected by a PEG chain.

Preferably, a weight-average molecular weight (Mw) of the 4-Arm PEG-acrylate is 5,000-40,000, including but not limited to: 5,000; 5,100; 5,500; 6,000; 8,000; 10,000; 12,000; 15,000; 16,000; 18,000; 19,000; and 20,000.

More preferably the weight-average molecular weight (Mw) of the 4-Arm PEG-acrylate is 5,000; 8,000; 12,000; 16,000; or 20,000.

Preferably, the weight-average molecular weight (Mw) of the SH-PEG-SH is 1,000-20,000, including but not limited to: 1,000; 1,200; 1,500; 1,600; 2,000; 3,000; 3,400; 5,000; 5,100; 5,500; 6,000; 8,000; 9,000; 10,000; 12,000; 15,000; 18,000; 19,000; and 20,000.

More preferably, the weight-average molecular weight (Mw) of the SH-PEG-SH is 1,000; 5,000; 10,000; 15,000; or 20,000.

Preferably, the target nucleic acid molecule is selected from the group consisting of Escherichia coli 23S ribosomal gene, cytokeratin 19 gene, HPV gene and a combination thereof.

It is understood that all conventional nucleic acid amplification methods in the art are applicable to the present application.

Preferably, the nucleic acid amplification reaction is selected from the group consisting of a loop-mediated isothermal amplification (LAMP), a recombinase polymerase amplification, a polymerase chain reaction (PCR), and a rolling circle amplification (RCA).

It is understood that conventional reagents for nucleic acid amplification in the field are all applicable to the present application, such as reagents for LAMP reaction, e.g., WarmStart LAMP 2× Master Mix (NEB) and LAMP Fluorescent Dye (NEB).

In some embodiments, the reagents include lysozyme, Proteinase K, random hexamers, polymerases (e.g., Phi29 DNA polymerase, Taq polymerase, Bst polymerase), transposases (e.g., Tn5), primers (e.g., P5 and P7 adapter sequences), ligase enzymes, deoxynucleotide triphosphates (dNTPs), buffers, or divalent cations.

Preferably, the fluorescent reagent is a fluorescent dye or fluorescent probe, and the fluorescent dye includes N,N-dimethyl-N′-[4-[(E)-(3-methyl-1,3-benzothiazol-2-ylidene)methyl]-1-phenylquinolin-1-ium-2-yl]-N′-propylpropane-1,3-diamine (SYBR Green I).

In some embodiments, when the nucleic acid amplification reaction is loop-mediated isothermal amplification (LAMP), the reagents used are WarmStart LAMP 2× Master Mix (NEB), LAMP Fluorescent Dye (NEB), and sterile water. These reagents are conventional reagents for LAMP reactions, which are commercially available and generally include buffers and sterile water.

The WarmStart LAMP 2× Master Mix (NEB) mixture contains Bst 2.0 WarmStart DNA Polymerase and WarmStart RTx reverse transcriptase, and its buffer includes 20 mM Tris-HCl, 10 mM (NH₄)₂SO₄, 50 mM KCl, 2 mM MgSO₄, and 0.1% Tween® 20.

In some embodiments, when the nucleic acid amplification reaction is rolling circle amplification (RCA), the reagents used are 10×phi29 DNA Polymerase Reaction Buffer (NEB), dNTPs, SYBR Green dye, and sterile water.

The 10×phi29 DNA Polymerase Reaction Buffer (NEB) mixture contains phi29 DNA Polymerase, and its buffer includes 50 mM Tris-HCl, 10 mM MgCl₂, 10 mM (NH₄)₂SO₄, and 4 mM DTT.

In some embodiments, when the nucleic acid amplification reaction is polymerase chain reaction (PCR), the reagents used are PrimeSTAR Max Premix (2×), Eva Green dye, and sterile water.

The PrimeSTAR Max Premix (2×) mixture contains PrimeSTAR Max DNA Polymerase, with a buffer formulation of 2 mM Mg²⁺ and 0.4 mM dNTP.

These reagents are conventional and commercially available for nucleic acid amplification. Only the 4-arm PEG-acrylate, SH-PEG-SH, a primer specific to the target nucleic acid molecule, and the analyte are required to be added, the content of the target nucleic acid molecule can be detected.

Alternatively, the PEG hydrogel polymer can be replaced with PNIPAM hydrogel, PHEMA hydrogel, PAM hydrogel, or P(AMPS-AM) hydrogel, to detect the content of the target nucleic acid molecule.

One of ordinary skill in the art may select conventional commercially available reagents as needed.

It is understood that primers designed for different target nucleic acid molecules enable the detection of any target nucleic acid.

In some embodiments, when the nucleic acid amplification reaction is loop-mediated isothermal amplification (LAMP) and the target is E. coli, the primer comprises sequences shown in SEQ ID NOs: 1-4. Preferably, the primer comprises sequences shown in SEQ ID NOs: 1-6. SEQ ID NOs: 1-4 (i.e., outer primers and inner primers) are essential for the amplification, while SEQ ID NO:5 and SEQ ID NO:6 are loop primers, which are optional. The addition of loop primers, i.e., six primers in total, can improve the amplification efficiency. One of ordinary skill in the art may adjust the primer composition or select other suitable primers as needed. The corresponding fluorescent dye is LAMP Fluorescent Dye (NEB), which is a nucleic acid intercalating dye that enables the real-time detection of LAMP reactions by fluorescence measurement. This dye exhibits weak or negligible fluorescence in its free state but emits strong fluorescence upon binding to double-stranded DNA (dsDNA).

Preferably, the forward outer primer consists of SEQ ID NO:1.

Preferably, the forward inner primer consists of SEQ ID NO:2.

Preferably, the reverse outer primer for the E. coli 23S ribosomal gene comprises the nucleic acid sequence of SEQ ID NO:3.

Preferably, the reverse inner primer for the E. coli 23S ribosomal gene comprises the nucleic acid sequence of SEQ ID NO:4.

Preferably, the forward loop primer for the E. coli 23S ribosomal gene comprises the nucleic acid sequence of SEQ ID NO:5.

Preferably, the reverse loop primer for the E. coli 23S ribosomal gene comprises the nucleic acid sequence of SEQ ID NO:6.

(F3):
SEQ ID NO: 1
5′-GGCGTTAAGTTGCAGGGTAT-3′.
(FIP):
SEQ ID NO: 2
5'-CGGTTCGGTCCTCCAGTTAGTGTTTTCCCGAAACCCGGTGATCT-
3′.
(B3):
SEQ ID NO: 3
5′-TCACGAGGCGCTACCTAA-3′.
(BIP):
SEQ ID NO: 4
5′-TAGCGGATGACTTGTGGCTGGTTTTTCGGGGAGAACCAGCTATC-
3′.
(LoopF):
SEQ ID NO: 5
5′-ACCTTCAACCTGCCCATG-3′.
(LoopB):
SEQ ID NO: 6
5′-GTGAAAGGCCAATCAAACC-3′.

In some embodiments, when the nucleic acid amplification reaction is loop-mediated isothermal amplification (LAMP) and the target is E. coli, the primers used are those listed in SEQ ID NO: 1 to SEQ ID NO:4, and preferably SEQ ID NO: 1 to SEQ ID NO: 6. When the fluorescent probes are used instead of fluorescent dyes, the corresponding probe sequences are: FAM-CGGTTCGGTCCTCCAGTTAGTGTTTTCCCGAAACCCGGTGATCT (SEQ ID NO: 2) and GGACCGAACCG-Blackhole Quencher 1 (SEQ ID NO:10). The FAM is the fluorophore, and the Blackhole Quencher 1 (BHQ-1) is the quenching fluorophore.

The described LAMP fluorescent probe pair consists of a fluorophore-labeled inner primer (FIP, SEQ ID NO:2) and a quencher-labeled probe complementary to a short sequence at the 5′ end of the fluorophore-labeled primer. The working principle of the LAMP probe is as follows. In the initial state, the fluorescent probe and the quencher probe are hybridized through complementary pairing, resulting in fluorescence quenching. When the reaction begins, the fluorescent probe and quencher probe denature and separate. The fluorescent probe then participates in the amplification reaction as an inner primer and becomes incorporated into the amplification product. Subsequently, the quencher probe can no longer quench the fluorescence of the fluorescent probe that has been incorporated into the product. After the reaction is completed, the temperature decreases, and unincorporated fluorescent probes re-hybridize with the quencher probes, restoring fluorescence quenching, while the fluorescent probes incorporated into amplification products generate detectable fluorescence signals.

In some embodiments, when the nucleic acid amplification reaction is rolling circle amplification (RCA) and the target is the cytokeratin 19 gene, the primer used is the sequence shown in SEQ ID NO:7.

Preferably, the primer for the cytokeratin 19 gene comprises the nucleic acid sequence of SEQ ID NO:7.

(Primer):
SEQ ID NO: 7
CCTGTTCCGTTCTTCCTTCA.

One of ordinary skill in the art may adjust or select other suitable primers as needed. The corresponding fluorescent dye includes but is not limited to SYBR Green. SYBR Green is a fluorescent dye that binds to double-stranded DNA (dsDNA). When not bound to dsDNA, SYBR Green exhibits very weak fluorescence, and once binded to dsDNA, its fluorescence intensity increases significantly. In RCA reactions, SYBR Green dye is used to monitor the amplification process in real time.

The chemical name of SYBR Green I is N,N-dimethyl-N′-[4-[(E)-(3-methyl-1,3-benzothiazol-2-ylidene)methyl]-1-phenylquinolin-1-ium-2-yl]-N′-propylpropane-1,3-diamine, with the molecular formula C₃₂H₃₇N₄S⁺.

In some embodiments, when the nucleic acid amplification reaction is rolling circle amplification (RCA) and the target is the cytokeratin 19 gene, the primer used is the sequence of SEQ ID NO:7. When fluorescent probes are used instead of fluorescent dyes, the corresponding fluorescent probe sequence is FAM-CGACAATGCCCGTCTGGCTGCAG (SEQ ID NO:11).

This probe contains fluorophores (FAM) but no quenchers. After the RCA reaction is completed, the RCA probe is added to the system, followed by reaction at 37° C. for 30 min. After the reaction, the system is rinsed with PBS buffer to remove unbound probes. Fluorescence imaging is then performed to calculate the copy number of the target nucleic acid molecule.

In some embodiments, when the nucleic acid amplification reaction is PCR and the target is the HPV gene, the primers used are the sequences of SEQ ID NO:8 and SEQ ID NO:9.

Preferably, the forward primer for the HPV gene comprises the nucleic acid sequence of SEQ ID NO:8.

Preferably, the reverse primer for the HPV gene comprises the nucleic acid sequence of SEQ ID NO:9.

(FP):
SEQ ID NO: 8
5′-CTCTTTGGCTGCCTAGTGAG-3′.
(RP):
SEQ ID NO: 9
5′-GCGTGCAACATATTCATCCG-3′.

One of ordinary skill in the art may adjust or select other suitable primers as needed. The corresponding fluorescent dye includes but is not limited to EvaGreen dye, which is a green fluorescent nucleic acid dye used for real-time quantitative PCR (qPCR). EvaGreen dye per se exhibits negligible fluorescence but emits strong fluorescence after being bound to double-stranded DNA (dsDNA).

In some embodiments, when the nucleic acid amplification reaction is PCR and the target is the HPV gene, the primers used are the sequences of SEQ ID NO:8 and SEQ ID NO:9. One of ordinary skill in the art may adjust or select other suitable primers as needed. The corresponding fluorescent probe sequences are FAM-CTCTTTGGCTGCCTAGTGAG (SEQ ID NO:8) and AGGCAGCCAAAGAG-Blackhole Quencher 1 (SEQ ID NO:12). FAM is the fluorophore, and Blackhole Quencher 1 is the quencher. The mechanism of this probe is identical to that of the LAMP probe described earlier.

In a second aspect, the present application provides a nucleic acid absolute quantification kit, which contains the aforementioned nucleic acid absolute quantification system, and a container configured to hold the nucleic acid absolute quantification system. The container is used in conjunction with a temperature control module compatible with the container to achieve nucleic acid amplification. The kit is used in conjunction with an optical system,
which includes a light sheet fluorescence microscope (LSFM) and instructions.

The nucleic acid absolute quantification system is as described above.

In some embodiments, the four-arm polyethylene glycol acrylate (4-arm PEG-acrylate) and dithiol polyethylene glycol (SH-PEG-SH) in the kit spontaneously polymerize into a hydrogel at room temperature. The hydrogel entraps and separates the target nucleic acid molecules, ensuring that amplification occurs at the original location without product diffusion. The double-stranded DNA (dsDNA) binds to fluorescent dyes, forming distinct fluorescent amplification spots for quantitative detection. Alternatively, quantitative detection is achieved by observing fluorescence differences before and after amplification when fluorescent probes are used.

The kit employs any container capable of holding the hydrogel system (e.g., PCR tubes) as miniature reaction vessels. After mixing the components of the nucleic acid absolute quantitative detection system with the analyte in the container, the hydrogel spontaneously polymerizes, trapping the analyte that is rich in genetic material within the micropores while allowing primers and reagents to diffuse through the porous structure of the hydrogel. The container is then directly transferred to a temperature control module for nucleic acid amplification. The amplified nucleic acid remains confined within the porous hydrogel structure, enabling in situ amplification.

In some embodiments, the container compatible with the hydrogel system includes a PCR tube for PCR thermocyclers and a container compatible with constant temperature modules for rolling circle amplification (RCA).

Unlike PCR, which requires precise temperature control using a thermocycler, RCA reactions can be performed under constant temperature conditions (e.g., in a water bath). For the RCA reaction, the container can be used with a water bath.

Replacing traditional glass microscope slides or incubation chamber gaskets with PCR tubes eliminates issues such as bubble formation and time-consuming manual handling that untrained users often encounter when spreading hydrogels into thin layers on glass slides or chamber gaskets.

The porous hydrogel formed within the container has a three-dimensional structure. After amplification, double-stranded DNA (dsDNA) binds to fluorescent dyes to form distinct fluorescent amplification spots. During light-sheet fluorescence microscopy (LSFM) scanning, a single fluorescent spot may appear in adjacent image frames. To address this, spots at the same position across consecutive frames are counted as a single spot during quantification.

Containers compatible with the hydrogel system include quartz tubes, transparent 96-well plate single-well tubes, and PCR tubes. The PCR tubes are designed for use with PCR thermocyclers, which include single tubes, tube strips, tubes with different cap types (dome, flat, flat/frosted, or no cap) and tubes of various colors.

More preferably, the PCR tubes are transparent.

Common PCR tube capacities: 0.2 mL, 0.5 mL, 100 μL, and 200 μL. 8-tube strips are available in 0.1 mL and 0.2 mL formats.

Centrifuge tubes (e.g., 200 μL tubes) with specifications identical or similar to PCR tubes may also be used, provided they are compatible with PCR thermocyclers. Such tubes fall within the scope of “PCR tubes” as defined here.

The optical system includes a light sheet fluorescence microscope (LSFM).

In some embodiments, a light sheet fluorescence microscope (LSFM) is employed and modified to count fluorescent spots within the container. This eliminates the complex washing steps for the hydrogel and the water-in-oil treatment to quantify spots, as each micropore in the hydrogel functionally mimics a droplet in the oil-water system. In the present disclosure, the hydrogel replaces the traditional oil-water emulsion.

For in situ amplification within the container, if the entire hydrogel is illuminated with excitation light, fluorescent spots would overlap. Therefore, the LSFM is used herein, where a thin laser light sheet excites fluorescence only in a single focal plane, and spots outside this plane remain unexcited, eliminating overlap.

The light source includes a line-shaped light source capable of generating a light sheet that excites fluorescence only at one plane of the sample.

During light-sheet scanning, a single fluorescent spot may appear across adjacent image frames. For accurate counting, spots detected at identical spatial coordinates in consecutive frames are treated as a single entity to avoid overcounting.

The counting and quantification of the fluorescent signal through the amplification sites are described herein. Absolute quantification can be achieved via direct counting, hemocytometer-like counting and Poisson distribution counting. The direct counting assumes that the number of fluorescent spots is equal to the copy number of the target nucleic acid, and the initial copy number of the target nucleic acid is calculated based on the sample volume. The hemocytometer-like counting extrapolates the total fluorescent spots in the hydrogel by analyzing the number of a subset volume of the fluorescent spots and a ratio of the representative subset volume to the full system volume, and the initial copy number of the target nucleic acid is calculated based on the sample volume. The Poisson distribution counting applies Poisson distribution formula to quantify nucleic acid concentration, which can directly calculate the concentration of the target nucleic acid in the original sample, enabling absolute quantification without requiring calibration standards.

$$Concentration = - ln ( \frac{{N}_{neg}}{N} ) / {V}_{droplet} ; \\ C = - ln ( \frac{{N}_{neg}}{N} ) ; or \\ C = ln ( N ) - ln ( {N}_{neg} ) ;$$

where Concentration refers to the concentration of the target nucleic acid molecule in the original sample; C represents the average number of target copies per microcompartment; N_{neg} represents the number of negative microcompartments; N represents the total number of droplets; Pr(n) denotes the probability of a microcompartment containing n target copies; E represents a proportion of empty microcompartments; and V_{droplet} is the volume of a microcompartment.

In a third aspect, the present application provides a nucleic acid absolute quantification method, comprising:
preparing the aforementioned nucleic acid absolute quantification system; performing nucleic acid amplification on a target nucleic acid molecule using the nucleic acid absolute quantification system to obtain an amplification product; conducting fluorescence imaging on the amplification product, and counting fluorescent bright spots; and calculating a copy number of the target nucleic acid molecule based on the number of the fluorescent bright spots.

By constructing a hydrogel-based nucleic acid amplification system, the porous structure of the hydrogel uniformly confines target nucleic acid molecules from the test sample within pores. Amplification and fluorescence imaging of the entire system are performed, and the initial copy number of the target nucleic acid in the sample is calculated directly from the number of fluorescent spots. This achieves absolute quantification without requiring additional physical structures or calibration curves, significantly reducing costs and simplifying workflows.

Preferably, the nucleic acid amplification reaction includes loop-mediated isothermal amplification (LAMP), recombinase polymerase amplification (RPA), polymerase chain reaction (PCR), and rolling circle amplification (RCA).

Conventional fluorescence analysis methods are applicable to this application. For example, the software hAmplicon_count-master can be used to count amplification spots and quantify fluorescence signals. Absolute quantification can be achieved through direct counting, hemocytometer-like counting, or Poisson distribution analysis, which further enables calculation of the target nucleic acid concentration in the original sample without standard curves.

The direct counting equates the number of fluorescent spots to the copy number of the target nucleic acid, with the initial copy number calculated from the sample volume.

The hemocytometer-like counting extrapolates the total fluorescent spots in the hydrogel by analyzing the number of a subset volume of the fluorescent spots and a ratio of the representative subset volume to the full system volume, and the initial copy number of the target nucleic acid is calculated based on the sample volume.

The Poisson distribution counting calculates the probability Pr(n) that each microcompartment in the hydrogel contains n copies of the target nucleic acid. If the average number of target copies per microcompartment is C, then:

$$P r ( n ) = \frac{{C}^{n} {e}^{- C}}{n !} .$$

For a fixed C, n=0 is input, then the probability of a microcompartment being empty is: Pr(0)=e^{-c}.

For a large number of microcompartments, the observed proportion of empty microcompartments (E) serves as an unbiased estimator of Pr(0):

$$ E = {e}^{- C} ; and \\ C = - ln ( E ) .$$

C (average copies per microcompartment) divides by the microcompartment volume to obtain copies per microliter (Concentration):

$$Concentration = \frac{C}{{V}_{compartment}} .$$

The above equations are combined to obtain:

$$Concentration = \frac{- ln ( E )}{{V}_{compartment}} .$$

The following equation is obtained according to definition:

$$E = \frac{{N}_{neg}}{N} ;$$

where N_{neg} is the number of negative (empty) microcompartments, and N is the total number of microcompartments.

The above equations are combined to obtain:

$$Concentration = - ln ( \frac{{N}_{neg}}{N} ) / {V}_{droplet} ; \\ C = - ln ( \frac{{N}_{neg}}{N} ) ; or \\ C = ln ( N ) - ln ( {N}_{neg} ) ;$$

where concentration represents a concentration of a target nucleic acid in the original sample; C represents an average target copies per microcompartment; N_{neg} represents the number of negative microcompartments; N represents a total number of microcompartments; Pr(n) represents a probability of a microcompartment containing n target copies; E represents a proportion of empty microcompartments; and V_{droplet} represents a volume of a single microcompartment.

Compared with the prior art, the present disclosure has at least the following advantages.

The present application introduces a novel hydrogel-based nucleic acid amplification system that utilizes the inherent porous structure of the hydrogel to uniformly confine target nucleic acid molecules within the porous structure. In addition, amplification and fluorescence imaging are performed on the entire hydrogel matrix, enabling direct calculation of the initial target copy number based on the number of fluorescent spots in the hydrogel. This approach achieves absolute quantification without requiring additional physical partitioning or labor-intensive standard curve calibration, which significantly reduces operational costs and simplifies processes.

### BRIEF DESCRIPTION OF THE DRAWINGS

FIG. 1A illustrates comparison among hydrogels formed by monomers of different molecular weights in terms of shape and size of fluorescent spots;

FIG. 1B illustrates comparison among hydrogels formed by monomers of different molecular weights in terms of shape and size of fluorescent spots;

FIG. 2A depicts distribution and diffusion of amplification spots in hydrogels with different monomer mass ratios according to an embodiment of the present disclosure;

FIG. 2B presents a bar chart correlating monomer mass ratios with fluorescent spot radii according to an embodiment of the present disclosure;

FIG. 3 shows an optical pathway diagram of a light-sheet fluorescence microscope (LSFM) according to an embodiment of the present disclosure;

FIG. 4 displays a physical image of the LSFM according to an embodiment of the present disclosure according to an embodiment of the present disclosure;

FIG. 5 schematically illustrates slicing of a post-amplification hydrogel using a laser sheet in the LSFM according to an embodiment of the present disclosure;

FIG. 6 shows a quantification result of nucleic acid amplification spots in a single hydrogel slice according to an embodiment of the present disclosure;

FIG. 7 presents a copy number detection result of Example 1;

FIG. 8 shows consistency analysis between a hydrogel loop-mediated isothermal amplification (LAMP) and a digital droplet PCR (ddPCR) of Example 1;

FIG. 9 shows consistency analysis between a hydrogel rolling circle amplification (RCA) and the ddPCR of Example 2;

FIG. 10 shows consistency analysis between a hydrogel polymerase chain reaction (PCR) and the ddPCR of Example 3;

FIGS. 11A-11E show consistency analysis between the hydrogel LAMP and the ddPCR of Example 4, where A-E demonstrate quantification effect for hydrogels with varying monomer mass ratios (4Arm-PEG-AC:SH-PEG-SH=1:10, 3:7, 1:1, 7:3, 10:1);

FIG. 12 is a bar chart showing relationships between monomer ratios of hydrogels (4Arm-PEG-AC:SH-PEG-SH=16:11, 1:10, 3:7, 1:1, 7:3, 10:1) with fluorescent spot radii according to an embodiment of the present disclosure;

FIG. 13 shows a consistency result between the hydrogel LAMP and the ddPCR of Example 5;

FIG. 14 shows a consistency result between the hydrogel LAMP and the ddPCR of Example 6;

FIG. 15 shows a consistency result between the hydrogel LAMP and the ddPCR of Example 7;

FIG. 16 shows a consistency result between the hydrogel LAMP and the ddPCR of Example 8;

FIG. 17 shows a consistency result between the hydrogel LAMP and the ddPCR of Example 9;

FIG. 18 is a receiver operating characteristic (ROC) curve showing comparison between experimental results and clinical tuberculosis diagnoses of Example 10;

FIG. 19 shows a consistency result between the hydrogel LAMP and the ddPCR of Example 11; and

FIG. 20 shows a consistency result between the hydrogel LAMP and the ddPCR of Example 12.

### DETAILED DESCRIPTION OF EMBODIMENTS

To further elaborate on the technical approaches and effects of this application, the present application will be further described with reference to specific embodiments and accompanying drawings. It is understood that the detailed implementations described herein are intended to explain the application rather than to restrict the scope of the application.

Unless otherwise specified, the experiments in the following examples are performed in accordance with the method or conditions recited in the literatures, or as recommended by the manufacturer, and the used reagents or instruments are commercially-available.

Conventional nucleic acid extraction methods are applicable to this application. For example, in specific embodiments, samples containing target nucleic acids are collected, and cells/bacteria are lysed using a nucleic acid extraction kit to release nucleic acids into solution. For example, for E. coli 23S ribosomal DNA quantification, 10 μL of sample is mixed with 90 μL DNA Extraction Solution 1.0 (Biosearch Technologies), placed at 65° C. for 1 min, and then placed at 95° C. for 1 min to extract DNA of the sample.

Primers targeting specific regions are designed to enable quantitative analysis of the target nucleic acid.

While the example below uses a loop-mediated isothermal amplification (LAMP) for E. coli 23S gene, the method is equally applicable to other amplification techniques, e.g., polymerase chain reaction (PCR), recombinase polymerase amplification (RPA), and rolling circle amplification (RCA).

Primer sequences and concentrations are listed as follows:

23S forward outer primer
SEQ ID NO: 1
(F3, 0.2 μM): 5′-GGCGTTAAGTTGCAGGGTAT-3′.
23S reverse outer primer
SEQ ID NO: 3
(B3, 0.2 μM): 5′-TCACGAGGCGCTACCTAA-3′.
23S forward inner primer (FIP, 1.6 μM):
SEQ ID NO: 2
5'-CGGTTCGGTCCTCCAGTTAGTGTTTTCCCGAAACCCGGTGATCT-3'.
23S reverse inner primer
SEQ ID NO: 4
(BIP, 1.6 μM):
5′-TAGCGGATGACTTGTGGCTGGTTTTTCGGGGAGAACCAGCTATC-3′.
23S forward loop primer
SEQ ID NO: 5
(LoopF, 0.4 μM): 5′-ACCTTCAACCTGCCCATG-3′.
23S reverse loop primer
SEQ ID NO: 6
(LoopB, 0.4 μM): 5′-GTGAAAGGCCAATCAAACC-3′.

In this application, a hydrogel-based LAMP reaction system contains WarmStart LAMP 2× Master Mix (NEB), a primer mixture, a LAMP fluorescent dye (NEB), four-arm polyethylene glycol acrylate (4-arm-PEG-AC), a dithiol polyethylene glycol (SH-PEG-SH), a sample DNA, and sterile water.

Hydrogel formation includes the following steps: the mixed reaction system is sealed in a 200 μL centrifuge tube (PCR tube) at room temperature within 5 min to form the hydrogel.

In this application, a 200 μL centrifuge tube is used as a PCR tube for the subsequent amplification.

The amplification is conducted as follows: the centrifuge tube is transferred to a PCR thermocycler for LAMP amplification (65° C. for 25 min).

Result analysis is described below. After the amplification, fluorescent signals are scanned using a light-sheet fluorescence microscope, and the software hAmplicon_count-master is employed for spot counting.

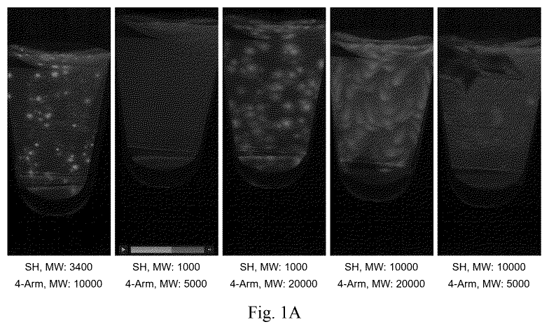

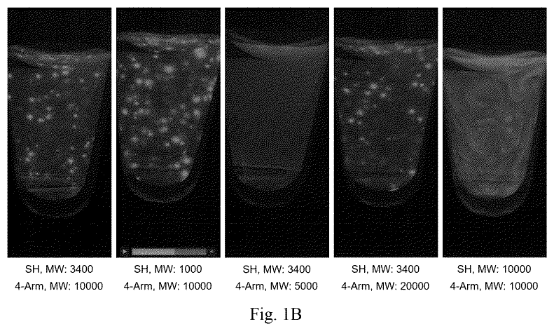

Conventional hydrogel monomers, such as hydroxyethyl methacrylate (HEMA) and polyethylene glycol diacrylate (PEGDA), which are commonly used in UV crosslinking, thermal crosslinking, or catalyst-driven crosslinking, can be polymerized in the presence of an initiator of HMPP under UV light or in the presence of APS under high temperature. These polymerization conditions may damage the nucleic acid amplification system, reduce amplification efficiency, and diminish fluorescence intensity. For instance, UV polymerization degrades nucleic acids, leading to underestimated quantitative results. Similarly, temperature-responsive hydrogels like agarose or gelatin, whose sol-gel transitions depend on temperature, conflict with the temperature requirements of nucleic acid amplification, making it challenging to establish stable hydrogel-based amplification systems. Therefore, this application prioritizes developing a mild hydrogel polymerization strategy. Initial attempts to perform amplification in gentler agarose hydrogels failed to produce observable fluorescent spots. In contrast, thiol-containing polymers spontaneously form hydrogels at room temperature without initiators, which does not affect the nucleic acid amplification reaction system. Therefore, a hydrogel system formed by 4-arm polyethylene glycol acrylate (4Arm-PEG-AC) and dithiol polyethylene glycol (SH-PEG-SH) is selected herein. Nine hydrogel variants are synthesized by combining 4Arm-PEG-AC with varying molecular weights (Mw: 5,000; 10,000; 20,000) with SH-PEG-SH with different molecular weights (Mw: 1,000; 3,400; 10,000). Morphology and size of fluorescent spots in the hydrogel are observed, as shown in FIGS. 1A and 1B, which identifies that the hydrogel system formed by 4Arm-PEG-AC (Mw: 10,000) and SH-PEG-SH (Mw: 3,400) is optimal.

While the other eight hydrogel formulations exhibit variations in fluorescent spot clarity, none showed spot fusion. Under non-fusion conditions, all fluorescent spots are successfully imaged and quantified using light-sheet fluorescence microscopy.

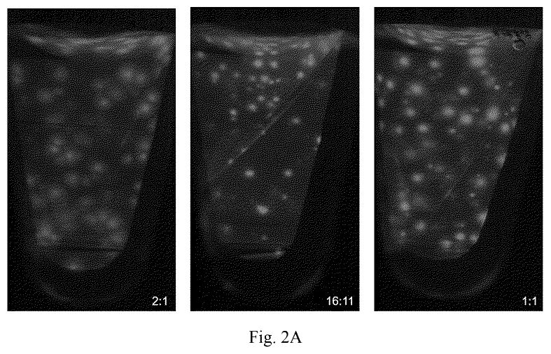

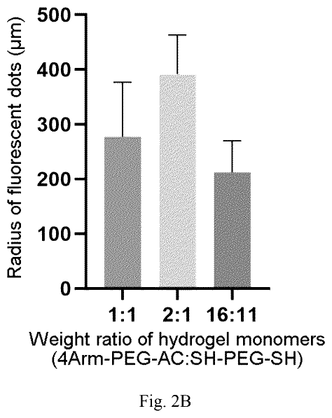

The mass ratio of the two monomers critically determines the gelation speed, porosity, and pore size of the hydrogel, which in turn influence nucleic acid amplification efficiency and speed, and fluorescence diffusion. To identify the optimal monomer ratio, LAMP amplification is performed on hydrogels with varying monomer proportions, and fluorescence intensity and diffusion after the amplification are analyzed. FIG. 2A illustrates amplification patterns of hydrogels with monomer ratios (4Arm-PEG-AC:SH-PEG-SH) of 2:1, 16:11, and 1:1, showing the amplification spots and diffusion of these hydrogels. FIG. 2B presents a bar chart correlating monomer mass ratios with fluorescent spot radii. It shows that the ratio of 16:11 exhibits minimal diffusion, thereby establishing that the mass ratio (4Arm-PEG-AC:SH-PEG-SH) of 16:11 is optimal. Although slight diffusion occurred at ratios of 2:1 and 1:1, fluorescent spots remain discrete (no fusion) and can be recorded by a light-sheet fluorescence microscopy, enabling reliable quantification.

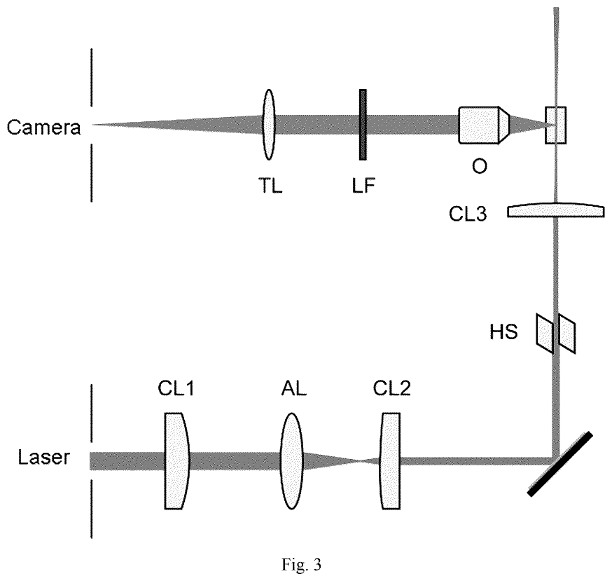

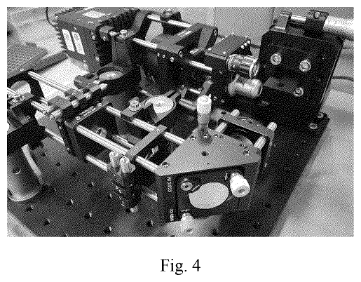

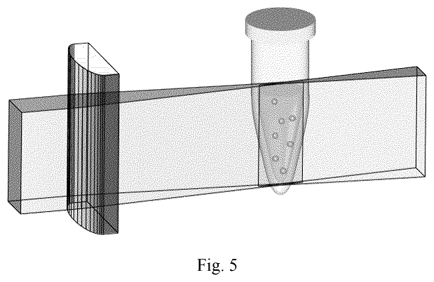

In this application, a light-sheet fluorescence microscope can be assembled based on the described optical configuration. Within the illumination path (as shown in FIG. 3), the laser is collimated into a Gaussian beam approximately 3.5 mm in diameter. This beam then passes through a layered structure consisting of an aspheric lens (f=8 mm) and two cylindrical lenses (CL1 and CL2, f=20 mm and 12.7 mm), converting the circular beam into an elliptical profile with a shortened optical path. The beam expansion ratios along the z-axis and y-axis are ×0.4 and ×1.6, respectively, forming an elliptical beam measuring 5.6×1.4 mm. Behind the layered structure, an orthogonally positioned adjustable mechanical slit (0-8 mm) further trims the beam to control the height and thickness of the light sheet. As illustrated in FIG. 4, the laser passes through an illumination cylindrical lens (CL3, f=50 mm) to generate a large and wide light sheet for optical sectioning of the hydrogel layer by layer. In the detection path, a ×2 infinity-corrected wide-field system (Leica Plan Fluor 4×/0.13 objective+Thorlabs TTL100 tube lens) is constructed perpendicular to the illumination path to collect fluorescence signals. After the amplification, the sample tube is mounted on a motorized stage via a tube clamp. During signal acquisition, the tube and hydrogel rapidly pass through the scanning light sheet along the z-axis. FIG. 5 schematically illustrates slicing of a post-amplification hydrogel using a laser sheet. A high-speed camera (Phantom Miro C320) captures images from the light-sheet plane at hundreds of frames per second.

In this application, the software hAmplicon_count-master is developed using Python and OpenCV for fluorescent spot counting. After scanning, the sample data is stored as a video file. Upon importing the video into the software, the dimensions, frame rate, and encoding format of the video are extracted. A new output video matching these parameters is created as an output result of the software. Each frame of the input video is sequentially read, converted to grayscale, denoised via median blur, and analyzed using the Hough Circle Transform to detect fluorescent amplification spots. The Hough Circle Transform operates under the principle that every non-zero pixel in the image could lie on a potential circle. By accumulating votes in a coordinate plane and applying a cumulative weight, a circle is localized. The equation for a circle in Cartesian coordinates is:

$${( x - a )}^{2} + {( y - b )}^{2} = {r}^{2} ;$$

where (a, b) represents the center of the circle and r represents the radius, and the relationship can also be expressed as:

$$x = a + r cos ( θ ) ; and \\ y = b + r sin ( θ ) .$$

Therefore, within the three-dimensional abr coordinate system, a single point uniquely defines a circle. In the Cartesian xy coordinate system, all circles passing through a specific point correspond to a cone in the abr coordinate system. All points lying on the same circle in the xy system share identical circle equations and thus map to the same point in the abr coordinate system. Consequently, this point in the abr coordinate system accumulates intersections from curves representing all pixels on the circle. By evaluating the accumulated intersection count in the abr coordinate system, circles are identified when the count exceeds a predefined threshold. After Hough circle detection, the detected circles are overlaid onto the original image. The software then determines whether a circle in the current frame corresponds to the same amplification spot as in the previous frame, enabling accurate counting. The final count results are annotated on the original video frames, synchronized with the original video frames, and exported as a new video.

While LAMP is used as an example in this application, the method is not limited to LAMP and is applicable to other nucleic acid amplification reactions, including but not limited to PCR, RPA, and RCA.

### (1) 25 μL Hydrogel Nucleic Acid Amplification Reaction System

The reaction system includes 12.5 μL of Warm Start LAMP 2× Master Mix (NEB), 2.5 μL of a primer mixture, 0.5 μL of a LAMP Fluorescent Dye (NEB), 1.92 mg of 4-Arm-PEG-AC, 1.32 mg of SH-PEG-SH, 2.5 μL of a purified or unpurified sample DNA and 2 μL sterile water.

When multiple samples are processed, many systems are required. However, manually adding individual components to each reaction tube is time- and labor-intensive. A more efficient approach is described below. A total volume of shared components required for all reactions is calculated, and a bulk master mixture is prepared followed by aliquoting.

For instance, a bulk master mix is prepared by adding a total volume of the shared components, followed by uniform mixing, aliquoting, and addition of a sample DNA and remaining monomers.

### (2) Hydrogel Formation

After thorough mixing, the prepared reaction system is sealed within a hydrogel-compatible container and allowed to gel at room temperature within minutes. In some preferred embodiments, gelation occurs within 2-5 min.

The sealing is typically achieved by closing the lid to prevent contamination caused by the post-amplification nucleic acid. This aligns with standard PCR protocols requiring sealed amplification. If lids are not closed, the system can be sealed by mineral oil.

### (3) Nucleic Acid Amplification

The sealed container is transferred to a temperature control module for amplification. For LAMP, the reaction conditions are 65° C. for 25 min. The temperature control module may include a PCR thermocycler or a constant-temperature module (e.g., water bath) suitable for isothermal reactions like RCA.

### (4) Result Interpretation

After the amplification, fluorescence signals are scanned using a light-sheet fluorescence microscope. The software hAmplicon_count-master quantifies target nucleic acids by counting fluorescent spots, where each spot corresponds to one target molecule. The total spot count directly reflects the target copy number.

In the present application, according to the three-dimensional spatial fluorescent signals of nucleic acid amplification spots, derived from light-sheet fluorescence microscopy scanning, the software hAmplicon_count-master is used to count amplification spots and quantify fluorescence signals. Absolute quantification can be achieved through direct counting, hemocytometer-like counting, or Poisson distribution analysis, which further enables calculation of the target nucleic acid concentration in the original sample without standard curves.

The direct counting equates the number of fluorescent spots to the copy number of the target nucleic acid, with the initial copy number calculated from the sample volume.

The hemocytometer-like counting extrapolates the total fluorescent spots in the hydrogel by analyzing the number of a subset volume of the fluorescent spots and a ratio of the representative subset volume to the full system volume, and the initial copy number of the target nucleic acid is calculated based on the sample volume.

The Poisson distribution counting calculates the probability Pr(n) that each microcompartment in the hydrogel contains n copies of the target nucleic acid. If the average number of target copies per microcompartment is C, then:

$$Pr ( n ) = \frac{{C}^{n} {e}^{- C}}{n !} .$$

For a fixed C, n=0 is input, then the probability of a microcompartment being empty is: Pr(0)=e⁻.

For a large number of microcompartments, the observed proportion of empty microcompartments (E) serves as an unbiased estimator of Pr(0):

$$E = {e}^{- C} ; and \\ C = - ln ( E ) .$$

C (average copies per microcompartment) divides by the microcompartment volume to obtain copies per microliter (Concentration):

$$Concentration = \frac{C}{{V}_{compartment}} .$$

The above equations are combined to obtain:

$$Concentration = \frac{- ln ( E )}{{V}_{compartment}} .$$

The following equation is obtained according to definition:

$$E = \frac{{N}_{neg}}{N} ;$$

where N_{neg} is the number of negative (empty) microcompartments, and N is the total number of microcompartments.

The above equations are combined to obtain:

$$Concentration = - ln ( \frac{{N}_{neg}}{N} ) / {V}_{droplet} ; \\ C = - ln ( \frac{{N}_{neg}}{N} ) ; or \\ C = ln ( N ) - ln ( {N}_{neg} ) ;$$

where concentration represents a concentration of a target nucleic acid in the original sample.

Specifically, this application validates the nucleic acid absolute quantitative detection system and method using E. coli 23S ribosomal gene, cytokeratin 19 (CK19) gene, and human papillomavirus (HPV) gene.

Digital droplet PCR (ddPCR), referenced in the examples, is a molecular biology technique based on PCR. It partitions the PCR mixture into numerous discrete reaction units using tiny droplets. Each tiny droplet contains a small amount of template DNA for independent amplification. This method offers high precision and sensitivity for analyzing low-copy targets, detecting rare mutations, and quantifying gene expression. Commercial systems include a ddPCR system provided by Bio-Rad.

Abbreviations in the examples are listed as follows.

NIPAM: N-isopropylacrylamide; KPS: Potassium persulfate (K₂S₂O₈); APS: Ammonium persulfate ((NH₄)₂S₂O₈); MBAA: N,N′-methylenebisacrylamide; HEMA: Hydroxyethyl methacrylate; EGDMA: Ethylene glycol dimethacrylate; TMEDA: tetramethylethylenediamine; AM: Acrylamide; PEGDA: Polyethylene glycol diacrylate; HMPP: 2-hydroxy-2-methylpropiophenone; AMPS: 2-acrylamido-2-methylpropanesulfonic acid; and BIS: N,N′-methylenebisacrylamide.

Reagents and materials used in the examples are commercially available, and E. coli strain (ATCC: 25922) is preserved in our laboratory.

All chemicals and reagents were analytical grade and used without further purification. All experiments are conducted in ultrapure water.

In the examples, the copy number is calculated using the following formula:

Copy number=(nucleic acid concentration/nucleic acid molecular weight)×Avogadro's constant.

Equipment and reagents used in the examples are listed below:

PCR thermocycler: Bio-Rad; light-sheet microscope: Thorlabs; LAMP fluorescent dye: purchased from New England Biolabs (NEB); Sybr Green dye: purchased from Takara; and EvaGreen dye: purchased from Biotium.

Primers and probes used herein are synthesized by Shanghai Sangon Biotechnology Co., Ltd.

Buffers used herein are all commercially available.

For example, WarmStart LAMP 2× Master Mix (NEB) mixture solution contains Bst 2.0 Warm Start DNA Polymerase and Warm Start RTx reverse Transcriptase, and its buffer contains 20 mM Tris-HCl, 10 mM (NH₄)₂SO₄, 50 mM KCl, 2 mM MgSO₄, and 0.1% Tween®20.

10×phi29 DNA Polymerase Reaction Buffer (NEB) mixture solution contains phi29 DNA Polymerase, and its buffer contains 50 mM Tris-HCl, 10 mM MgCl₂, 10 mM (NH₄)₂SO₄, and 4 mM DTT.

PrimeSTAR Max Premix (2×) mixture solution contains PrimeSTAR Max DNA Polymerase, and its buffer contains 2 mM Mg²⁺ and 0.4 mM dNTPs.

The present disclosure is further described with reference to specific examples.

Technical solutions in Examples 1-3 were validated using a droplet digital PCR (ddPCR) system.

### Example 1

In this example, a hydrogel LAMP system was employed for quantitative analysis of the E. coli 23S ribosomal gene.

Primers (shown in SEQ ID NO:1-SEQ ID NO:6) targeting the E. coli 23S gene were designed. 10 μL of E. coli bacteria solution (ATCC: 25922) was mixed with 90 μL of DNA Extraction Solution 1.0 (Biosearch Technologies), followed by standing at 65° C. for 1 min and 95° C. for 1 min to extract a sample DNA. The hydrogel LAMP reaction system (25 μL) included 12.5 μL of WarmStart LAMP 2× Master Mix (NEB), 2.5 μL of 10× primer mixture (SEQ ID NOs: 1-6), 0.5 μL of LAMP Fluorescent Dye (NEB), 1.92 mg of 4-Arm-PEG-AC (Mw: 10,000), 1.32 mg of SH-PEG-SH (Mw: 3,400), 2.5 μL of the sample DNA and 2 μL of sterile water. Systems containing 1-13 target copies (labeled CN1-CN13) were prepared, with a system free of the sample DNA as a control group (NTC).

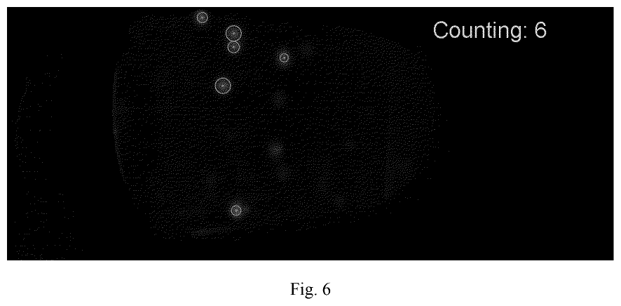

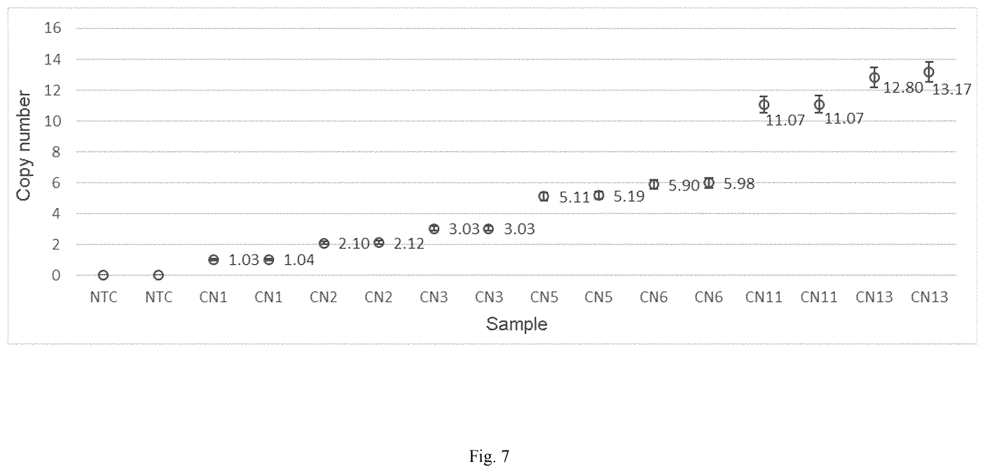

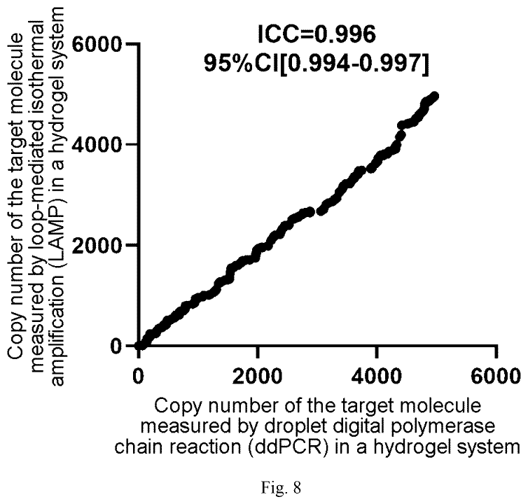

The mixed system was sealed in a 200 μL centrifuge tube followed by standing at room temperature for 5 min to form a hydrogel. The hydrogel was transferred to a thermocycler for LAMP amplification (65° C. for 25 min). After the amplification, the sample was mounted on a motorized stage via a tube clamp. Fluorescent signals were scanned using a light-sheet fluorescence microscope, and hAmplicon_count-master software was used to count amplification spots. The above treatments were conducted on each system. FIG. 6 exemplified quantitative results of a single hydrogel slice. FIG. 7 displayed the copy number detection results, with the NTC group as a blank group. The horizontal axis represented theoretical values, and the vertical axis showed measured values, confirming accurate detection from 1 copy (CN1) to 13 copies (CN13) with high resolution. Moreover, as shown in FIG. 8, the experimental result was compared with an analysis result of the same sample by using a ddPCR system, which demonstrated excellent consistency and yielded an intraclass correlation coefficient of 0.996.

The validation for the ddPCR system involved designing primers targeting the E. coli 23S ribosomal gene. The fluorescent probe used herein was FAM-CCCGAAACCCGGTGATCT-Blackhole Quencher 1 (SEQ ID NO: 13). 10 μL of E. coli bacteria solution (ATCC: 25922) was mixed with 90 μL of DNA Extraction Solution 1.0 (Biosearch Technologies), followed by standing at 65° C. for 6 min and 95° C. for 4 min to extract a sample DNA. The ddPCR reaction system (20 μL) included 10 μL of QX200 ddPCR Supermix for Probes (no dUTP) (Bio-Rad), 1.8 μL of a mixture of primer (SEQ ID NO:1, SEQ ID NO:3) and probe (SEQ ID NO:13), 2 μL of a sample DNA and 6.2 μL of sterile water. After droplets were generated, the droplet mixture was transferred to a thermocycler for amplification under the following conditions: 95° C. for 10 min; 94° C. for 30 s and 58° C. for 60 s for 40 cycles; and 98° C. for 10 min. After the amplification, droplets were analyzed using a droplet reader to quantify fluorescence signals.

### Example 2

In this example, a hydrogel RCA system was employed for quantitative analysis of the cytokeratin 19 (CK19) gene.

A primer (shown in SEQ ID NO:7) targeting the cytokeratin 19 (CK19) gene was designed. The hydrogel RCA reaction system (25 μL) included 2.5 μL of 10×phi29 DNA Polymerase Reaction Buffer (NEB), 0.25 μL of a recombinant albumin, 5 μL of dNTPs, 2.5 μL of 10× primer (SEQ ID NO:7) mixture, 1 μL of phi29 DNA Polymerase (NEB), 1.92 mg of 4-Arm-PEG-AC (Mw: 10,000), 1.32 mg of SH-PEG-SH (Mw: 3,400), 2.5 μL of a sample DNA, 0.25 μL of SYBR Green dye, and sterile water added to a final volume of 25 μL. The mixed system was sealed in a 200 μL centrifuge tube followed by standing at room temperature for 5 min to form a hydrogel. The hydrogel was transferred to a thermocycler for RCA amplification (30° C. for 120 min). After the amplification, the sample was mounted on a motorized stage via a tube clamp. Fluorescent signals were scanned using a light-sheet fluorescence microscope, and hAmplicon_count-master software was used to count amplification spots.

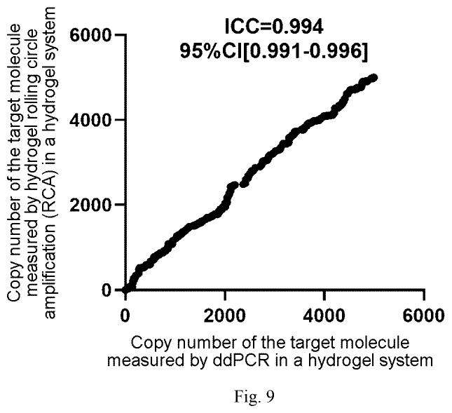

The experimental result was compared with an analysis result of the same sample by using a ddPCR system, which demonstrated excellent consistency and yielded an intraclass correlation coefficient of 0.994, as shown in FIG. 9.

### Example 3

In this example, a hydrogel PCR system was employed for quantitative analysis of the HPV DNA

Primers (shown in SEQ ID NO:8-SEQ ID NO:9) targeting the HPV DNA was designed. The hydrogel PCR reaction system (25 μL) included 12.5 μL of PrimeSTAR Max Premix (2×), 2.5 μL of 10× primer mixture (SEQ ID NO:8, SEQ ID NO:9), 0.5 μL of Eva Green dye, 1.92 mg of 4-Arm-PEG-AC (Mw: 10,000), 1.32 mg of SH-PEG-SH (Mw: 3,400), 2.5 μL of a sample DNA, and sterile water added to a final volume of 25 μL. The mixed system was sealed in a 200 μL centrifuge tube followed by standing at room temperature for 5 min to form a hydrogel. The hydrogel was transferred to a thermocycler for PCR amplification (conditions: 98° C. for 20 s; 98° C. for 10 s and 72° C. for 30 s for 30 cycles; and 72° C. for 2 min). After the amplification, the sample was mounted on a motorized stage via a tube clamp. Fluorescent signals were scanned using a light-sheet fluorescence microscope, and hAmplicon_count-master software was used to count amplification spots.

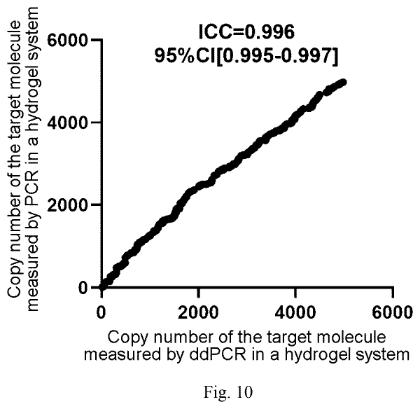

The experimental result was compared with an analysis result of the same sample by using a ddPCR system, which demonstrated excellent consistency and yielded an intraclass correlation coefficient of 0.996, as shown in FIG. 10.

### Example 4

In this example, hydrogel LAMP systems prepared with different hydrogel monomer mass ratios were employed for quantitative analysis of the E. coli 23S ribosomal gene.

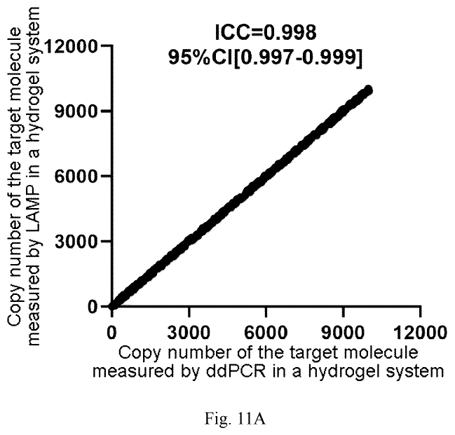

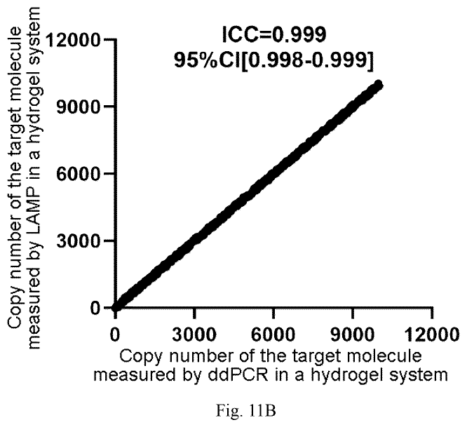

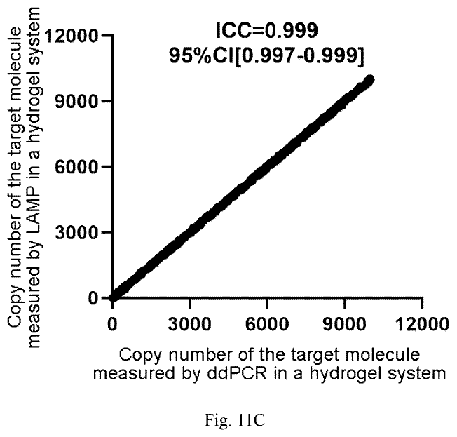

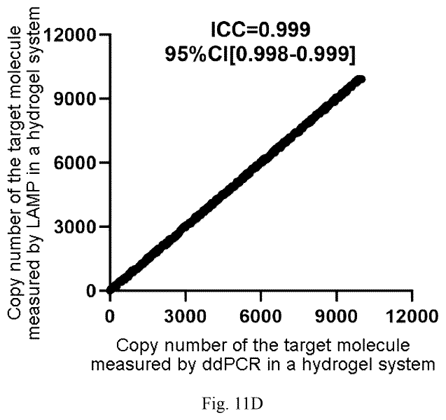

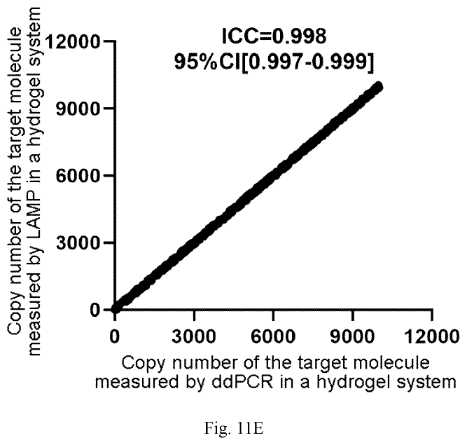

Primers (shown in SEQ ID NO: 1-SEQ ID NO:6) targeting the E. coli 23S gene were designed. 10 μL of E. coli bacteria solution (ATCC: 25922) was mixed with 90 μL of DNA Extraction Solution 1.0 (Biosearch Technologies), followed by standing at 65° C. for 1 min and 95° C. for 1 min to extract a sample DNA. The hydrogel LAMP reaction system (25 μL) included 12.5 μL of WarmStart LAMP 2× Master Mix (NEB), 2.5 μL of 10× primer mixture (SEQ ID NOs: 1-6), 0.5 μL of LAMP Fluorescent Dye (NEB), 4Arm-PEG-AC (Mw: 10,000) and SH-PEG-SH (Mw: 3,400) at varying weight ratios (i.e., 1:10:0.12 mg 4-Arm-PEG-AC+3.12 mg SH-PEG-SH; 3:7:0.97 mg 4-Arm-PEG-AC+2.27 mg SH-PEG-SH; 1:1:1.62 mg 4-Arm-PEG-AC+1.62 mg SH-PEG-SH; 7:3:2.27 mg 4-Arm-PEG-AC+0.97 mg SH-PEG-SH; and 10:1:3.12 mg 4-Arm-PEG-AC+0.12 mg SH-PEG-SH), 2.5 μL of the sample DNA and 2 μL of sterile water. Systems containing different target copies were prepared, with a system free of the sample DNA as a control group (NTC). The mixed system was sealed in a 200 μL centrifuge tube followed by standing at room temperature for 2 min to form a hydrogel. The hydrogel was transferred to a thermocycler for LAMP amplification (65° C. for 10 min). The duration of 10 min was determined based on preliminary experiments showing that fluorescent spot sizes after 10 min of amplification matched those observed in Example 1 after 25 min. After the amplification, the sample was mounted on a motorized stage via a tube clamp, and fluorescent signals were scanned using a light-sheet fluorescence microscope. Fluorescent amplification spots were counted using a hAmplicon_count-master software. Quantitative results for hydrogels with monomer mass ratios of 1:10, 3:7, 1:1, 7:3, and 10:1 were shown in FIG. 11.

A-E in FIG. 11 corresponded to the quantitative result of respective monomer mass ratios (4-Arm-PEG-AC:SH-PEG-SH=1:10, 3:7, 1:1, 7:3, 10:1). Experimental results were compared with analysis results of the same sample by using a ddPCR system, which demonstrated excellent consistency and yielded intraclass correlation coefficients of 0.998-0.999.

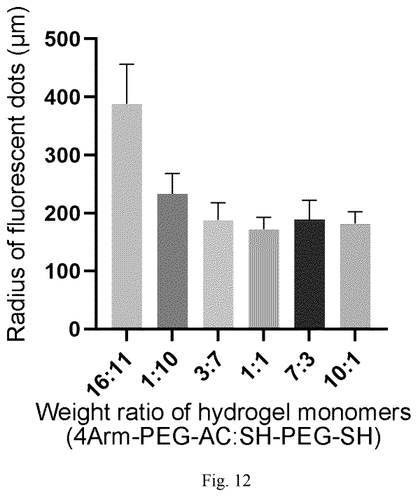

Compared to Examples 1-3, hydrogels crosslinked with varying monomer weight ratios exhibited reduced fluorescence diffusion ranges (as shown in FIG. 12). Additionally, this example achieved shorter gelation times and significantly reduced amplification durations, and enhanced resistance to interference. Detailed data were summarized in Table 1.

TABLE 1

| | Hydrogel monomer weight ratios | | | |
|---|---|---|---|---|
| | (4Arm-PEG-AC:SH-PEG-SH) | | | |
| | 16:11 | 16:11 | 16:11 | 1:10 |
| Examples | 1 | 2 | 3 | 4 |
| Reaction time (min) | 5 | 5 | 5 | 2 |
| Radius of fluorescent dot (μm) | 388 | 388 | 388 | 233 |
| | Hydrogel monomer weight ratios | | | |
| | (4Arm-PEG-AC:SH-PEG-SH) | | | |
| | 3:7 | 1:1 | 7:3 | 10:1 |
| Examples | 4 | 4 | 4 | 4 |
| Reaction time (min) | 2 | 2 | 2 | 2 |
| Radius of fluorescent dot (μm) | 188 | 172 | 189 | 182 |

### Example 5

In this example, hydrogel LAMP systems prepared with monomers of varying weight-average molecular weights (Mw) were employed for quantitative analysis of the E. coli 23S ribosomal gene.

Primers (shown in SEQ ID NO: 1-SEQ ID NO:6) targeting the E. coli 23S gene were designed. 10 μL of E. coli bacteria solution (ATCC: 25922) was mixed with 90 μL of DNA Extraction Solution 1.0 (Biosearch Technologies), followed by standing at 65° C. for 1 min and 95° C. for 1 min to extract a sample DNA. The hydrogel LAMP reaction system (25 μL) included 12.5 μL of WarmStart LAMP 2× Master Mix (NEB), 2.5 μL of 10× primer mixture, 0.5 μL of LAMP Fluorescent Dye (NEB), 1.92 mg of 4Arm-PEG-AC (Mw: 5,000; 8,000; 12,000; 16,000; and 20,000), 1.32 mg of SH-PEG-SH (Mw: 1,000; 5,000; 10,000; 15,000; and 20,000), 2.5 μL of the sample DNA and 2 μL of sterile water. Systems containing different target copies were prepared, with a system free of the sample DNA as a control group (NTC). The mixed system was sealed in a 200 μL centrifuge tube followed by standing at room temperature for 2 min to form a hydrogel. The hydrogel was transferred to a thermocycler for LAMP amplification (65° C. for 25 min). After the amplification, the sample was mounted on a motorized stage via a tube clamp, and fluorescent signals were scanned using a light-sheet fluorescence microscope. Fluorescent amplification spots were counted using a hAmplicon_count-master software. Quantitative analysis effects of the present Example were similar with those of Example 4.

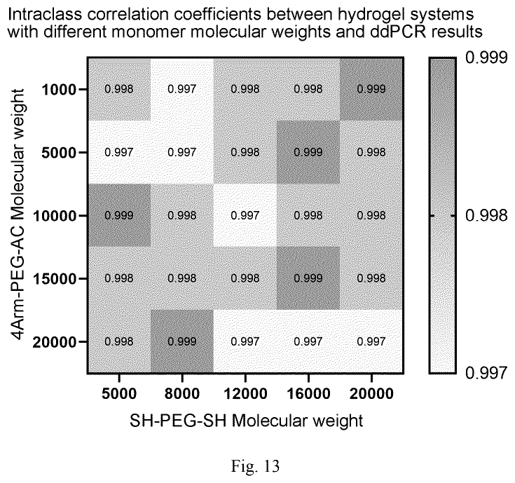

As shown in FIG. 13, experimental results were compared with analysis results of the same sample by using a ddPCR system, which demonstrated excellent consistency and yielded intraclass correlation coefficients of 0.997-0.999.

### Example 6

In this example, hydrogel LAMP systems prepared with different polyethylene glycol acrylate (PEG-AC) monomers were employed for quantitative analysis of the E. coli 23S ribosomal gene.

Primers (shown in SEQ ID NO:1-SEQ ID NO:6) targeting the E. coli 23S gene were designed. 10 μL of E. coli bacteria solution (ATCC: 25922) was mixed with 90 μL of DNA Extraction Solution 1.0 (Biosearch Technologies), followed by standing at 65° C. for 1 min and 95° C. for 1 min to extract a sample DNA. The hydrogel LAMP reaction system (25 μL) included 12.5 μL of WarmStart LAMP 2× Master Mix (NEB), 2.5 μL of 10× primer mixture (SEQ ID NOs: 1-6), 0.5 μL of LAMP Fluorescent Dye (NEB), different PEG-Acs with Mw of 10,000 (i.e., 3.84 mg of PEG diacrylate (AC-PEG-AC), 2.56 mg of 3-arm-PEG-AC, 1.92 mg of 4-arm-PEG-AC, and 0.96 mg of 8-arm-PEG-AC), 1.32 mg of SH-PEG-SH (Mw: 3,400), 2.5 μL of the sample DNA and 2 μL of sterile water. Systems containing different target copies were prepared, with a system free of the sample DNA as a control group (NTC). The mixed system was sealed in a 200 μL centrifuge tube followed by standing at room temperature for 5 min to form a hydrogel. The hydrogel was transferred to a thermocycler for LAMP amplification (65° C. for 25 min).

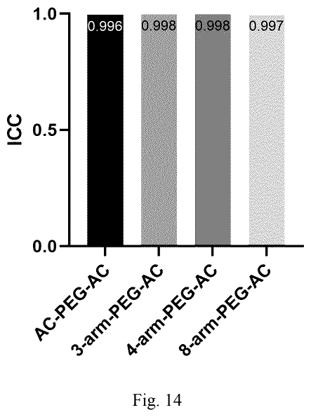

After the amplification, the samples were mounted on a motorized stage via a tube clamp, and fluorescent signals were scanned using a light-sheet fluorescence microscope. Fluorescent amplification spots were counted using a hAmplicon_count-master software. Quantitative results for the hydrogels were shown in FIG. 14. In this Example, the PEG-Ac was AC-PEG-AC, 3-arm-PEG-AC, 4-arm-PEG-AC, or 8-arm-PEG-AC. Experimental results were compared with analysis results of the same sample by using a ddPCR system, which demonstrated excellent consistency and yielded intraclass correlation coefficients of 0.996, 0.998, 0.998, and 0.997, respectively.

### Example 7

In this example, hydrogel LAMP systems prepared with different polyethylene glycol maleimide (MAL-PEG) monomers were employed for quantitative analysis of the E. coli 23S ribosomal gene.

Primers (shown in SEQ ID NO:1-SEQ ID NO:6) targeting the E. coli 23S gene were designed. 10 μL of E. coli bacteria solution (ATCC: 25922) was mixed with 90 AL of DNA Extraction Solution 1.0 (Biosearch Technologies), followed by standing at 65° C. for 1 min and 95° C. for 1 min to extract a sample DNA. The hydrogel LAMP reaction system (25 μL) included 12.5 μL of WarmStart LAMP 2× Master Mix (NEB), 2.5 μL of 10× primer mixture (SEQ ID NOs: 1-6), 0.5 μL of LAMP Fluorescent Dye (NEB), a MAL-PEG with Mw of 10,000 (i.e., 3.84 mg of bismaleimide polyethylene glycol (MAL-PEG-MAL), 2.56 mg of 3-arm-PEG-MAL, 1.92 mg of 4-arm-PEG-MAL, or 0.96 mg of 8-arm-PEG-MAL), 1.32 mg of SH-PEG-SH (Mw: 3,400), 2.5 μL of the sample DNA and 2 μL of sterile water. Systems containing different target copies were prepared, with a system free of the sample DNA as a control group (NTC). The mixed system was sealed in a 200 μL centrifuge tube followed by standing at room temperature for 5 min to form a hydrogel. The hydrogel was transferred to a thermocycler for LAMP amplification (65° C. for 25 min).

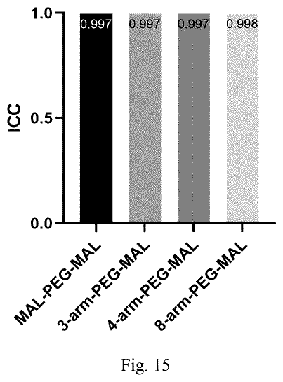

After the amplification, the samples were mounted on a motorized stage via a tube clamp, and fluorescent signals were scanned using a light-sheet fluorescence microscope. Fluorescent amplification spots were counted using a hAmplicon_count-master software. Quantitative results for the hydrogels were shown in FIG. 15. In this Example, the MAL-PEG was MAL-PEG-MAL, 3-arm-PEG-MAL, 4-arm-PEG-MAL, or 8-arm-PEG-MAL. Experimental results were compared with analysis results of the same sample by using a ddPCR system, which demonstrated excellent consistency and yielded intraclass correlation coefficients of 0.997, 0.997, 0.997, and 0.998, respectively.

### Example 8

In this example, hydrogel LAMP systems prepared with different polyethylene glycol thiol monomers were employed for quantitative analysis of the E. coli 23S ribosomal gene.

Primers (shown in SEQ ID NO:1-SEQ ID NO:6) targeting the E. coli 23S gene were designed. 10 μL of E. coli bacteria solution (ATCC: 25922) was mixed with 90 μL of DNA Extraction Solution 1.0 (Biosearch Technologies), followed by standing at 65° C. for 1 min and 95° C. for 1 min to extract a sample DNA. The hydrogel LAMP reaction system (25 μL) included 12.5 μL of WarmStart LAMP 2× Master Mix (NEB), 2.5 μL of 10× primer mixture, 0.5 μL of LAMP Fluorescent Dye (NEB), 1.92 mg of 4-arm-PEG-MAL (Mw: 10,000), a polyethylene glycol-thiol compound with a Mw of 3,400 (i.e., 1.32 mg of SH-PEG-SH or 0.65 mg of 4-arm-PEG-SH), 2.5 μL of the sample DNA and 2 μL of sterile water. Systems containing different target copies were prepared, with a system free of the sample DNA as a control group (NTC). The mixed system was sealed in a 200 μL centrifuge tube followed by standing at room temperature for 5 min to form a hydrogel. The hydrogel was transferred to a thermocycler for LAMP amplification (65° C. for 25 min).

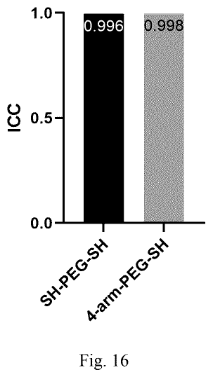

After the amplification, the samples were mounted on a motorized stage via a tube clamp, and fluorescent signals were scanned using a light-sheet fluorescence microscope. Fluorescent amplification spots were counted using a hAmplicon_count-master software. Quantitative results for the hydrogels were shown in FIG. 16. In this Example, the polyethylene glycol-thiol compound was SH-PEG-SH or 4-arm-PEG-SH. Experimental results were compared with analysis results of the same sample by using a ddPCR system, which demonstrated excellent consistency and yielded intraclass correlation coefficients of 0.996 and 0.998, respectively.

### Example 9

In this Example, different hydrogel LAMP systems were employed for the quantitative analysis of the E. coli 23S ribosomal gene. The components and their molecular weights (Mw) of the hydrogel were listed as follows: 4-arm-PEG-AC (Mw: 10,000), SH-PEG-SH (Mw: 3,400), HEMA (Mw: 130.14), EGDMA (Mw: 198.22), APS (Mw: 228.2), NIPAM (Mw: 113.16), K₂S₂O₈ (Mw: 270.32), MBAA (Mw: 154.17), AM (Mw: 71.08), PEGDA (Mw: 700), HMPP (Mw: 164.21), AMPS (Mw: 207.2), BIS (Mw: 154.17), and TMEDA (Mw: 116.20).

Primers (shown in SEQ ID NO:1-SEQ ID NO:6) targeting the E. coli 23S gene were designed. 10 μL of E. coli bacteria solution (ATCC: 25922) was mixed with 90 μL of DNA Extraction Solution 1.0 (Biosearch Technologies), followed by standing at 65° C. for 1 min and 95° C. for 1 min to extract a sample DNA.

The hydrogel LAMP reaction system (25 μL) included:
(1) 12.5 μL of WarmStart LAMP 2× Master Mix (NEB);
(2) 2.5 μL of 10× primer mixture (SEQ ID NO:1-SEQ ID NO:6);
(3) 0.5 μL of LAMP Fluorescent Dye (NEB);
(4) a PEG hydrogel system (1.92 mg of 4-arm-PEG-MAL and 1.32 mg of SH-PEG-SH), or a pHEMA hydrogel system (2.24 mg of HEMA, 0.1 mg of EGDMA, 0.02 mg of APS, and 0.02 mg of TMEDA), or a PNIPAM hydrogel system (1.04 mg of NIPAM, 0.05 mg K₂S₂O₈ and 0.01 mg of MBAA), or a PAM hydrogel system (2.5 mg of AM, 0.31 mg of PEGDA, and 0.03 mg of HMPP), or a PAM hydrogel system (2.38 mg of AM, 0.13 mg of BIS, 12.5 μg of APS and 12.5 μg of TEMED), or a P (AMPS-AM) hydrogel system (0.88 mg of AMPS, 1.76 mg of AM, 0.08 mg of BIS and 0.22 mg of HMPP);
(5) 2.5 μL of the sample DNA; and
(6) sterile water added to reach 25 μL.

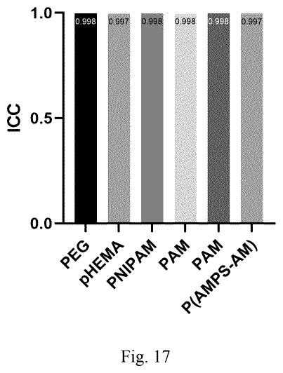

Systems containing different target copies were prepared, with a system free of the sample DNA as a control group (NTC). The mixed system was sealed in a 200 μL centrifuge tube followed by standing at room temperature for 5 min to form a hydrogel. The hydrogel was transferred to a thermocycler for LAMP amplification (65° C. for 25 min). After the amplification, the samples were mounted on a motorized stage via a tube clamp, and fluorescent signals were scanned using a light-sheet fluorescence microscope. Fluorescent amplification spots were counted using a hAmplicon_count-master software. Quantitative results for the hydrogels were shown in FIG. 17. For the PEG hydrogel system, the pHEMA hydrogel system, the PNIPAM hydrogel system, the PAM hydrogel system and the P(AMPS-AM) hydrogel system, experimental results were compared with analysis results of the same sample by using a ddPCR system, which demonstrated excellent consistency and yielded intraclass correlation coefficients of 0.998, 0.997, 0.998, 0.998 and 0.997, respectively.

### Example 10

In this example, a hydrogel-based LAMP system was applied to quantify CXCL10 mRNA in clinical whole-blood samples.

Total RNA was extracted from whole blood, and a hydrogel LAMP reaction system was prepared. The hydrogel LAMP reaction system (25 μL) included 12.5 μL of WarmStart LAMP 2× Master Mix (NEB), 2.5 μL of 10× primer mixture (SEQ ID NO: 14-SEQ ID NO:18): 0.5 μL of LAMP Fluorescent Dye (NEB), a PEG hydrogel system (1.92 mg of 4Arm-PEG-AC (Mw: 10,000) and 1.32 mg of SH-PEG-SH (Mw: 3,400)), 1 μL of M-MLV reverse transcriptase, 2.5 μL of the sample RNA, and sterile water added to 25 μL.

(SEQ ID NO: 14)
F3: 5′-GTCCACGTGTTGAGATCA-3′.
(SEQ ID NO: 15)
B3: 5′-GGGAAGTGATGGGAGAGG-3′.
FIP:
(SEQ ID NO: 16)
5′-TTCTTGATGGCCTTCGATTCTGGCTACAATGAAAAAGAAGGGTG-
3′.
BIP:
(SEQ ID NO: 17)
GAAAGCAGTTAGCAAGGAAAGGTCTTGGAAGCACTGCATCG-3′.
LB:
(SEQ ID NO: 18)
5′-GATCTCCTTAAAACCAGAGGG-3′.

The mixed system was sealed in a 200 μL centrifuge tube followed by standing at room temperature for 5 min to form a hydrogel. The hydrogel was transferred to a thermocycler for LAMP amplification (65° C. for 25 min). After the amplification, the samples were mounted on a motorized stage via a tube clamp, and fluorescent signals were scanned using a light-sheet fluorescence microscope. Fluorescent amplification spots were counted using a hAmplicon_count-master software.

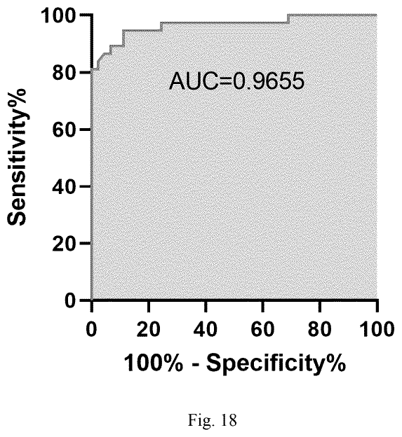

Experimental results were compared with clinical tuberculosis diagnostic results, which showed high consistency, achieving an area under the ROC curve of 0.9655 (as shown in FIG. 18).

### Example 11

In this example, a probe-based hydrogel PCR system was utilized for quantitative analysis of HPV DNA.

Primers (shown in SEQ ID NO:8-SEQ ID NO:9) targeting the HPV DNA was designed and a fluorescent probe sequence specific to the HPV gene was: FAM-CCACTGTCTACTTGCCTCCTGTCCCA-Blackhole Quencher 1 (SEQ ID NO:12).

The hydrogel PCR reaction system (25 μL) included 12.5 μL of TaqMan™ Fast Advanced Master Mix (2×), 2.5 μL of a 10× primer mixture (SEQ ID NO:8 and SEQ ID NO:9), 2.5 μL of a 10× fluorescent probe, 1.92 mg of 4Arm-PEG-AC (Mw: 10,000), 1.32 mg of SH-PEG-SH (Mw: 3,400), 2.5 μL of the sample DNA, and sterile water added to a final volume of 25 μL. The mixed system was sealed in a 200 μL centrifuge tube followed by standing at room temperature for 5 min to form a hydrogel. The hydrogel was transferred to a thermocycler for PCR amplification (conditions: 98° C. for 20 s; 98° C. for 10 s and 72° C. for 30 s for 30 cycles; and 72° C. for 2 min). After the amplification, the sample was mounted on a motorized stage via a tube clamp. Fluorescent signals were scanned using a light-sheet fluorescence microscope, and hAmplicon_count-master software was used to count amplification spots.

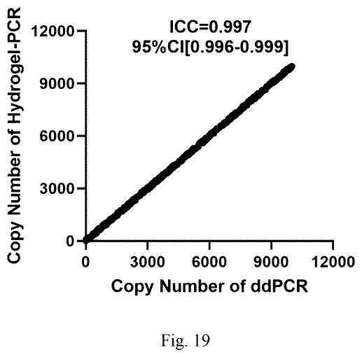

The experimental result was compared with an analysis result of the same sample by using a ddPCR system, which demonstrated excellent consistency and yielded an intraclass correlation coefficient of 0.997, as shown in FIG. 19.

### Example 12

In this example, a probe-based hydrogel LAMP system was employed for the quantitative analysis of the E. coli 23S ribosomal gene.

Fluorescent probe sequences targeting the E. coli 23S gene were designed as follows: FAM-CGGTTCGGTCCTCCAGTTAGTGTTTTCCCGAAACCCGGTGATCT (SEQ ID NO: 2); and GGACCGAACCG-Blackhole Quencher 1 (SEQ ID NO:10).

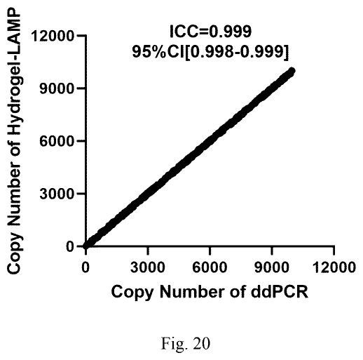

10 μL of E. coli bacteria solution (ATCC: 25922) was mixed with 90 μL of DNA Extraction Solution 1.0 (Biosearch Technologies), followed by standing at 65° C. for 1 min and 95° C. for 1 min to extract a sample DNA. The hydrogel LAMP reaction system (25 μL) included 12.5 μL WarmStart LAMP 2× Master Mix (NEB), 2.5 μL 10× primer-probe mixture (SEQ ID NOs: 1-6 and SEQ ID NO:10), 1.92 mg of 4Arm-PEG-AC (Mw: 10,000), 1.32 mg SH-PEG-SH (Mw: 3,400), 2.5 μL sample DNA and sterile water added to reach 25 μL. Systems containing different target copies were prepared, with a system free of the sample DNA as a control group (NTC). The mixed system was sealed in a 200 μL centrifuge tube followed by standing at room temperature for 5 min to form a hydrogel. The hydrogel was transferred to a thermocycler for LAMP amplification (65° C. for 25 min). After the amplification, the sample was mounted on a motorized stage via a tube clamp, and fluorescent signals were scanned using a light-sheet fluorescence microscope. Fluorescent amplification spots were counted using a hAmplicon_count-master software. Experimental results were compared with analysis results of the same sample by using a ddPCR system, which demonstrated excellent consistency and yielded an intraclass correlation coefficient of 0.999, as shown in FIG. 20.

In summary, the present application discloses a novel hydrogel-based nucleic acid amplification system that utilizes the inherently porous structure of hydrogels to uniformly confine target nucleic acid molecules within the porous structure. Moreover, amplification and fluorescence imaging are performed on the whole system, and the specific initial copy number of the target nucleic acid in the sample is calculated according to the number of the fluorescent spots in the hydrogel, thereby achieving absolute quantification, and eliminating the need for additional physical structures or standard curves. This approach significantly reduces costs and simplifies workflows compared to conventional methods.

The applicant declares that while the embodiments described herein illustrate the detailed implementation of the present application, the scope of the application is not limited to these specific examples. It is understood by one of ordinary skill in the art that modifications, equivalent substitutions of materials, additions of auxiliary components, or alternative methodologies fall within the protection scope of the present application.

What is claimed is:

1. A nucleic acid absolute quantification system, wherein the nucleic acid absolute quantification system is selected from the group consisting of a first system, a second system, a third system, a fourth system, a fifth system, a sixth system and a combination thereof;
wherein the first system comprises a polyethylene glycol acrylate compound containing two or more acrylate groups or a polyethylene glycol maleimide compound containing two or more maleimide groups, a polyethylene glycol-thiol compound containing two or more thiol groups, a primer for a target nucleic acid molecule, a nucleic acid amplification reagent, and a first fluorescent reagent;
wherein a mass ratio of the polyethylene glycol acrylate compound or the polyethylene glycol maleimide compound to the polyethylene glycol-thiol compound is 1-30:10-1;
the polyethylene glycol acrylate compound is selected from the group consisting of polyethylene glycol diacrylate (AC-PEG-AC), three-arm polyethylene glycol acrylate (3-arm-PEG-AC), four-arm polyethylene glycol acrylate (4-arm-PEG-AC), and eight-arm polyethylene glycol acrylate (8-arm-PEG-AC);
the polyethylene glycol maleimide compound is selected from the group consisting of bismaleimide polyethylene glycol (MAL-PEG-MAL), three-arm polyethylene glycol maleimide (3-arm-PEG-MAL), four-arm polyethylene glycol maleimide (4-arm-PEG-MAL), six-arm polyethylene glycol maleimide (6-arm-PEG-MAL), and eight-arm polyethylene glycol maleimide (8-arm-PEG-MAL); and
the polyethylene glycol-thiol compound is dithiol polyethylene glycol (SH-PEG-SH) or four-arm polyethylene glycol thiol (4-arm-PEG-SH);
the second system comprises N-isopropylacrylamide (NIPAM), potassium persulfate (KPS) as initiator, N,N-methylenebisacrylamide (MBAA) as crosslinker, the primer, the nucleic acid amplification reagent, and a second fluorescent reagent; wherein a mass ratio of the NIPAM to the KPS to the MBAA is 80-98:0.5-5:0.1-1;
the third system comprises hydroxyethyl methacrylate (HEMA), ethylene glycol dimethacrylate (EGDMA) as crosslinker, ammonium persulfate (APS) as initiator, tetramethylethylenediamine (TMEDA) as co-initiator, the primer, the nucleic acid amplification reagent, and a third fluorescent reagent; wherein a mass ratio of the HEMA to the EGDMA to the APS to the TMEDA is 70-95:0.5-5:0.1-1:0.1-1;
the fourth system comprises acrylamide (AM), polyethylene glycol diacrylate (PEGDA) as crosslinker, 2-hydroxy-2-methylpropiophenone (HMPP) as initiator, the primer, the nucleic acid amplification reagent, and a fourth fluorescent reagent; wherein a mass ratio of the AM to the PEGDA to the HMPP is 70-90:5-20:0.1-1;
the fifth system comprises acrylamide (AM), N,N′-methylenebisacrylamide (BIS), ammonium persulfate (APS) as initiator, tetramethylethylenediamine (TMEDA) as crosslinker, the primer, the nucleic acid amplification reagent, and a fifth fluorescent reagent; wherein a mass ratio of the AM to the BIS to the APS to the TMEDA is 152-228:8-12:0.1-2:0.1-2; and
the sixth system comprises 2-Acrylamido-2-methylpropane sulfonic acid (AMPS), acrylamide (AM), N,N′-methylenebisacrylamide (BIS) as crosslinker, 2-hydroxy-2-methylpropiophenone (HMPP) as initiator, the primer, the nucleic acid amplification reagent, and a sixth fluorescent reagent; wherein a mass ratio of the AMPS to the AM to the BIS to the HMPP is 1:2-10:1-3:2-4.

2. The nucleic acid absolute quantification system of claim 1, wherein the nucleic acid absolute quantification system is the first system, and the mass ratio of the polyethylene glycol acrylate compound or the polyethylene glycol maleimide compound to the polyethylene glycol-thiol compound is 1-30:10-1.

3. The nucleic acid absolute quantification system of claim 2, wherein a weight-average molecular weight of the polyethylene glycol acrylate compound or the polyethylene glycol maleimide compound is 5,000-40,000; and/or
a weight-average molecular weight of the polyethylene glycol-thiol compound is 1,000-20,000.

4. The nucleic acid absolute quantification system of claim 1, wherein the target nucleic acid molecule is selected from the group consisting of Escherichia coli 23S ribosomal gene, cytokeratin 19 gene, human papillomavirus (HPV) gene, and a combination thereof.

5. The nucleic acid absolute quantification system of claim 1, wherein the first fluorescent reagent, the second fluorescent reagent, the third fluorescent reagent, the fourth fluorescent reagent, the fifth fluorescent reagent and the sixth fluorescent reagent are each independently a fluorescent dye or a fluorescent probe; and
the fluorescent dye comprises N,N-dimethyl-N′-[4-[(E)-(3-methyl-1,3-benzothiazol-2-ylidene)methyl]-1-phenylquinolin-1-ium-2-yl]-N′-propylpropane-1,3-diamine.

6. The nucleic acid absolute quantification system of claim 1, wherein the nucleic acid amplification reagent is designed for loop-mediated isothermal amplification (LAMP), recombinase polymerase amplification (RPA), polymerase chain reaction (PCR), or rolling circle amplification (RCA).

7. The nucleic acid absolute quantification system of claim 4, wherein the target nucleic acid molecule is the Escherichia coli 23S ribosomal gene; the nucleic acid amplification reagent is designed for loop-mediated isothermal amplification; and
the primer comprises a forward outer primer consisting of SEQ ID NO: 1, a forward inner primer consisting of SEQ ID NO:2, a reverse outer primer consisting of SEQ ID NO: 3, and a reverse inner primer consisting of SEQ ID NO:4.

8. The nucleic acid absolute quantification system of claim 4, wherein the target nucleic acid molecule is the cytokeratin 19 gene; the nucleic acid amplification reagent is designed for rolling circle amplification; and the primer consists of a nucleotide sequence of SEQ ID NO:7.

9. The nucleic acid absolute quantification system of claim 4, wherein the target nucleic acid molecule is the HPV gene; the nucleic acid amplification reagent is designed for polymerase chain reaction; and the primer comprises a forward primer of SEQ ID NO:8 and a reverse primer of SEQ ID NO:9.

10. A nucleic acid absolute quantification kit, comprising:
the nucleic acid absolute quantification system of claim 1; and
a container configured to hold the nucleic acid absolute quantification system;
wherein the nucleic acid absolute quantification is in a hydrogel state; and the container is compatible with a temperature control module; and
the nucleic acid absolute quantification kit is compatible with light sheet fluorescence microscopy (LSFM).

11. The nucleic acid absolute quantification kit of claim 10, wherein the container comprises a PCR tube compatible with a PCR thermal cycler.

12. A nucleic acid absolute quantification method, comprising:
preparing the nucleic acid absolute quantification system of claim 1;
performing amplification on the target nucleic acid molecule using the nucleic acid absolute quantification system to obtain an amplification product;
conducting fluorescence imaging on the amplification product, and counting fluorescent bright spots; and
calculating a copy number of the target nucleic acid molecule based on the number of the fluorescent bright spots.

13. The nucleic acid absolute quantification method of claim 12, wherein the amplification is loop-mediated isothermal amplification, recombinase polymerase amplification, polymerase chain reaction, or rolling circle amplification.


SEQUENCE LISTING (18 sequences)

SEQ ID NO: 1 - DNA, 20 bp, synthetic construct
  source 1..20: mol_type = other DNA
ggcgttaagttgcagggtat

SEQ ID NO: 2 - DNA, 44 bp, synthetic construct
  source 1..44: mol_type = other DNA
cggttcggtcctccagttagtgttttcccgaaacccggtgatct

SEQ ID NO: 3 - DNA, 18 bp, synthetic construct
  source 1..18: mol_type = other DNA
tcacgaggcgctacctaa

SEQ ID NO: 4 - DNA, 44 bp, synthetic construct
  source 1..44: mol_type = other DNA
tagcggatgacttgtggctggtttttcggggagaaccagctatc

SEQ ID NO: 5 - DNA, 18 bp, synthetic construct
  source 1..18: mol_type = other DNA
accttcaacctgcccatg

SEQ ID NO: 6 - DNA, 19 bp, synthetic construct
  source 1..19: mol_type = other DNA
gtgaaaggccaatcaaacc

SEQ ID NO: 7 - DNA, 20 bp, synthetic construct
  source 1..20: mol_type = other DNA
cctgttccgttcttccttca

SEQ ID NO: 8 - DNA, 20 bp, synthetic construct
  source 1..20: mol_type = other DNA
ctctttggctgcctagtgag

SEQ ID NO: 9 - DNA, 20 bp, synthetic construct
  source 1..20: mol_type = other DNA
gcgtgcaacatattcatccg

SEQ ID NO: 10 - DNA, 11 bp, synthetic construct
  source 1..11: mol_type = other DNA
ggaccgaaccg

SEQ ID NO: 11 - DNA, 23 bp, synthetic construct
  source 1..23: mol_type = other DNA
cgacaatgcccgtctggctgcag

SEQ ID NO: 12 - DNA, 14 bp, synthetic construct
  source 1..14: mol_type = other DNA
aggcagccaaagag

SEQ ID NO: 13 - DNA, 18 bp, synthetic construct
  source 1..18: mol_type = other DNA
cccgaaacccggtgatct

SEQ ID NO: 14 - DNA, 18 bp, synthetic construct
  source 1..18: mol_type = other DNA
gtccacgtgttgagatca

SEQ ID NO: 15 - DNA, 18 bp, synthetic construct
  source 1..18: mol_type = other DNA
gggaagtgatgggagagg

SEQ ID NO: 16 - DNA, 44 bp, synthetic construct
  source 1..44: mol_type = other DNA
ttcttgatggccttcgattctggctacaatgaaaaagaagggtg

SEQ ID NO: 17 - DNA, 41 bp, synthetic construct
  source 1..41: mol_type = other DNA
gaaagcagttagcaaggaaaggtcttggaagcactgcatcg

SEQ ID NO: 18 - DNA, 21 bp, synthetic construct
  source 1..21: mol_type = other DNA
gatctccttaaaaccagaggg